SEQUENCE LISTING (26 sequences)

SEQ ID NO: 1 - DNA, 20 bp, Artificial Sequence
  forward primer
gacccttttcatccctcctc

SEQ ID NO: 2 - DNA, 20 bp, Artificial Sequence
  reverse WT/flox primer
tctcctcatgtccattgcag

SEQ ID NO: 3 - DNA, 20 bp, Artificial Sequence
  reverse KO primer
gaacttcagggtcagcttgc

SEQ ID NO: 4 - DNA, 20 bp, Artificial Sequence
  forward primer
gctacgccaacgacgccttc

SEQ ID NO: 5 - DNA, 24 bp, Artificial Sequence
  reverse primer
tgtcctgcagtgtcgtatagtcca

SEQ ID NO: 6 - protein, 17 aa, Artificial Sequence
  Aptide specific for the EDB (APTEDB)
CSSPIQGSWTWENGKWT

SEQ ID NO: 7 - protein, 8 aa, Artificial Sequence
  Aptide specific for the EDB (APTEDB)
WGIIRLEQ

SEQ ID NO: 8 - DNA, 23 bp, Artificial Sequence
  mouse RhoJ - forward primer
gggaagacctgcttgctgatgag

SEQ ID NO: 9 - DNA, 26 bp, Artificial Sequence
  mouse RhoJ - Reverse
gcagtgtcgtatagtccaagcaagtg

SEQ ID NO: 10 - DNA, 26 bp, Artificial Sequence
  mouse PDGFRalpha - Forward
cacggtgaaggacagtggagagtatg

SEQ ID NO: 11 - DNA, 25 bp, Artificial Sequence
  mouse PDGFRalpha - Reverse
cttcgtgcaagttgacagcttccag

SEQ ID NO: 12 - DNA, 23 bp, Artificial Sequence
  mouse PDGFRbeta - Forward
gctgctggagacactgggagatg

SEQ ID NO: 13 - DNA, 24 bp, Artificial Sequence
  mouse PDGFRbeta - Reverse
ggagtcacccaaggtacggttgtc

SEQ ID NO: 14 - DNA, 24 bp, Artificial Sequence
  mouse alpha-SMA - Forward
catcgtgggacgtcccagacatca

SEQ ID NO: 15 - DNA, 26 bp, Artificial Sequence
  mouse beta-SMA - Reverse
acagcctgaatagccacatacatggc

SEQ ID NO: 16 - DNA, 27 bp, Artificial Sequence
  mouse FSP-1 - Forward
cacaaatactcaggcaaagagggtgac

SEQ ID NO: 17 - DNA, 26 bp, Artificial Sequence
  mouse FSP-1 - Reverse
ctgttgctgtccaagttgctcatcac

SEQ ID NO: 18 - DNA, 26 bp, Artificial Sequence
  mouse GAPDH - Forward
gtcgtggagtctactggtgtcttcac

SEQ ID NO: 19 - DNA, 28 bp, Artificial Sequence
  mouse GAPDH - Reverse
gttgtcatatttctcgtggttcacaccc

SEQ ID NO: 20 - RNA, 19 nt, Artificial Sequence
  Control (SiC)
uagcgacuaaacacaucaa

SEQ ID NO: 21 - RNA, 19 nt, Artificial Sequence
  Human RhoJ (J0)
gcuguauaugaaagagaaa

SEQ ID NO: 22 - DNA, 19 bp, Artificial Sequence
  Human RhoJ (J1)
ccactgtgtttgaccacta

SEQ ID NO: 23 - DNA, 19 bp, Artificial Sequence
  Human RhoJ (J2)
agaaacctctcacttacga

SEQ ID NO: 24 - RNA, 19 nt, Artificial Sequence
  Human RhoJ (J3)
ucauagggacccagauuga

SEQ ID NO: 25 - RNA, 19 nt, Artificial Sequence
  Human RhoJ (J3)
ucagaaaggucucaaagcg

SEQ ID NO: 26 - DNA, 19 bp, Artificial Sequence
  Mouse RhoJ
gcgcagtgctacttggaat

(19) United States
(12) Patent Application Publication
(10) Pub. No.: US 2014/0370079 A1
(43) Pub. Date: Dec. 18, 2014

(54) METHOD FOR TARGETING VASCULAR RHOJ FOR INHIBITING TUMOR ANGIOGENESIS

(71) Applicant: Korea Advanced Institute of Science and Technology (KAIST), Daejeon (KR)

(72) Inventors: Gou Young KOH, Daejeon (KR); Chan Kim, Daejeon (KR); Hanseul Yang, Daejeon (KR); Won Do Heo, Daejeon (KR); Injune Kim, Daejeon (KR); Sangyong Jon, Daejeon (KR); Akiyoshi Uemura, Kobe (JP)

(73) Assignee: Korea Advanced Institute of Science and Technology (KAIST), Daejeon (KR)

(21) Appl. No.: 14/271,117

(22) Filed: May 6, 2014

Related U.S. Application Data
(60) Provisional application No. 61/835,900, filed on Jun. 17, 2013.

(51) Int. Cl. C12N 15/113; A61K 31/661; A61K 33/24; A61K 45/06

(52) U.S. Cl. CPC C12N 15/1137; A61K 45/06; A61K 31/661; A61K 33/24; C12N 2310/11; C12N 2320/31
USPC 424/450; 514/44 A; 424/649

(57) ABSTRACT

The present application describes a method of inhibiting tumor growth including contacting the tumor with a compound that inhibits activity of RhoJ protein.

### BACKGROUND OF THE INVENTION

1. Field of the Invention

The present application relates to a method of targeting RhoJ for suppressing tumor angiogenesis and inducing vascular disruption.

2. General Background and State of the Art

Vascular targeting therapies have been considered as one of the anti-cancer therapeutic options for the past decade. However, the survival benefit is usually only several months, depending on clinical conditions, because of intrinsic resistance and evasive mechanisms. Here, we show the potential of RhoJ blockade, through inhibition of angiogenesis and disruption of existing vessels in tumors, to be a powerful adjuvant option to complement and maximize the anti-cancer effects of conventional anti-angiogenic and vascular-disrupting agents. Our study provides a rationale for the development of specific inhibitors against RhoJ.

Tumor angiogenesis is a prerequisite for tumor progression (Ferrara and Alitalo, 1999; Hanahan and Folkman, 1996). The angiogenic switch is activated during tumor growth, and resulting tumor neovessels manage the O₂ and nutrient requirements as well as the clearance of CO₂ and metabolite in tumor tissue (Carmeliet and Jain, 2011; Hanahan and Folkman, 1996). Moreover, the tumor vasculature is one of main route of tumor cell metastasis to distant organs (Hanahan and Weinberg, 2011). Collectively, these observations imply that tumors cannot grow further and metastasize without sufficient blood supply. This inference has led to the development of various angiogenesis-inhibiting agents (AIAs) in the past decade (Ellis and Hicklin, 2008; Ferrara and Kerbel, 2005; Sennino and McDonald, 2012), many of which target vascular endothelial growth factor (VEGF) and its receptors and have proved to be effective in clinical practice (Carmeliet and Jain, 2011; Chung et al., 2010). In addition, ongoing drug development has focused on moderating other angiogenic pathways (Bono et al., 2013; Gerald et al., 2013; Koh et al., 2010; Sennino and McDonald, 2012; Tvorogov et al., 2010). Because current AIAs are inherently cytostatic and target newly growing tumor vasculature, they are more suited to tumor stabilization than to the regression of a bulky tumor (Ellis and Hicklin, 2008; Horsman and Siemann, 2006). Even after repeated cycles of AIA treatment, a substantial amount of preformed vasculature remains intact within the tumor. In addition, although it requires further investigation in clinics, several preclinical studies suggested that tumor cells could convert to a more aggressive phenotype with increased invasion and metastasis after the AIA treatment (Bergers and Hanahan, 2008; Casanovas et al., 2005; Ebos and Kerbel, 2011). Moreover, because VEGF and its receptors are expressed ubiquitously in normal tissues and in tumors, current AIAs produce adverse effects such as hypertension, proteinuria, and hemorrhage (Chen and Cleck, 2009; Kamba and McDonald, 2007). Therefore, it is important to better discern differences between tumor and normal vasculature in order to develop more selective and potent targeting strategies.

Rho GTPases have recently been discovered as fine-tuners of vascular morphogenesis and homeostasis (Bryan and d'Amore, 2007). Rho GTPases are considered as essential downstream targets of VEGF signaling in endothelial cells (ECs), and a well-controlled balance between different Rho GTPases governs almost all aspects of angiogenic processes such as EC migration, proliferation, extracellular matrix degradation, vascular morphogenesis, and vascular integrity (Beckers et al., 2010; Bryan and d'Amore, 2007; van der Meel et al., 2011). Although much remains to be unraveled about how different Rho GTPases are involved in angiogenesis and coordinate with each other, targeting Rho GTPases has become a promising strategy to enhance current anti-angiogenic treatment (van der Meel et al., 2011). One question to be answered is which Rho GTPase is the most promising anti-angiogenic target with high selectivity against tumor vasculature.

RhoJ is a Rho GTPase mainly expressed in ECs (Fukushima et al., 2011; Kaur et al., 2011; Takase et al., 2012; Yuan et al., 2011), and its expression is regulated by the endothelial transcription factor ERG in primary cultured human umbilical vein endothelial cells (HUVECs) (Yuan et al., 2011). Despite its vascular expression pattern, the importance of RhoJ in vascular biology is only beginning to emerge. A few recent papers have revealed that RhoJ is an important regulator of EC motility and tube morphogenesis in 3D matrices (Kaur et al., 2011; Yuan et al., 2011). During development, RhoJ is specifically expressed in the dorsal aorta and intersomitic vessels of mouse embryos as well as in the retinal vessels of the postnatal mouse (Fukushima et al., 2011; Kaur et al., 2011). RhoJ-deficient mice display delayed radial growth of retinal vasculature during postnatal development with increased vascular regression in the vascular front (Takase et al., 2012). Also, RhoJ-overexpressing mice attenuate the aberrant extraretinal vascular outgrowth in an oxygen-induced retinopathy model (Fukushima et al., 2011). Thus, RhoJ signaling primarily affects vessel remodeling via balancing neovessel formation and regression; however, the expression and function of RhoJ in tumor angiogenesis have not been elucidated thus far.

Because RhoJ is specifically expressed in ECs during development (Fukushima et al., 2011; Kaur et al., 2011; Leszczynska et al., 2011), we speculated that it is also expressed in the growing tumor vasculature. Here, we investigated the biological role and therapeutic relevance of targeting RhoJ in various solid tumor models.

### SUMMARY OF THE INVENTION

Current anti-angiogenic therapy is limited by its cytostatic nature and systemic side effects. To address these limitations, we have unveiled the role of RhoJ, an endothelial-enriched Rho GTPase, during tumor progression. RhoJ blockade provides a double assault on tumor vessels by both inhibiting tumor angiogenesis and disrupting the preformed tumor vessels, through the activation of the RhoA-ROCK (Rho kinase) signaling pathway in tumor endothelial cells, consequently resulting in a functional failure of tumor vasculatures. Moreover, enhanced anti-cancer effects were observed when RhoJ blockade was employed in concert with a cytotoxic chemotherapeutic, angiogenesis-inhibiting agent or vascular-disrupting agent. These results identify RhoJ blockade as a selective and effective therapeutic strategy for targeting tumor vasculature with minimal side effects.

The invention overcomes the above-mentioned problems, and provides a selective and effective therapeutic strategy for targeting tumor vasculature with minimal side effects.

In one aspect, the present invention is directed to a method of inhibiting tumor growth comprising contacting the tumor with a compound that inhibits activity of RhoJ protein. The tumor growth may occur in a subject, in which the subject may be a mammal and in particular a human being.

In another aspect, the present invention is directed to a method of inhibiting cancer metastasis in a subject comprising administering to the subject a compound that inhibits activity of RhoJ protein. The cancer metastasis may occur in a subject, in which the subject may be a mammal and in particular a human being.

In another aspect, the present invention is directed to a method of reducing tumor volume comprising contacting the tumor with a compound that inhibits activity of RhoJ protein. The tumor volume reduction may occur in a subject, in which the subject may be a mammal and in particular a human being.

In another aspect, the present invention is directed to a method of disrupting tumor vasculature comprising contacting the tumor with a compound that inhibits activity of RhoJ protein. The tumor vasculature disruption may occur in a subject, in which the subject may be a mammal and in particular a human being, and further wherein the tumor vasculature may be disrupted selectively.

In any of the above aspects, the compound may be an oligonucleotide complementary to a portion of RhoJ transcript, an antagonistic ligand of RhoJ, or a chemical compound that inhibits the activity of RhoJ.

In any of the above aspects, the inventive method may include further contacting the tumor with or administering to the subject, a compound that sequesters VEGF in combination with the compound that inhibits activity of RhoJ protein. The compound that sequesters VEGF may be preferably VEGF-trap.

In any of the above aspects, the inventive method may further include contacting the tumor with or administering to the subject, a vascular-disrupting agent (VDA) in combination with the compound that inhibits activity of RhoJ protein. The VDA may be preferably combretastatin-A4-phosphate (CA4P).

In any of the above aspects, the inventive method may further include contacting the tumor with or administering to the subject, a cytotoxic therapeutic agent in combination with the compound that inhibits activity of RhoJ protein. The cytotoxic therapeutic agent may be preferably cisplatin.

In any of the above aspects, the compound or agent may be included in a carrier.

In any of the above aspects, the carrier may be an aptide conjugated liposome.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1. RhoJ Is Highly Expressed in Tumor ECs during Tumor Progression. Unless otherwise denoted: Scale bars, 100 μm. Each group, n=5. Values are mean±SD. Dotted lines indicate the boundaries between the skin and tumor. (A) Images showing RhoJ expressions (green) in CD31⁺ tumor vessels of LLC and B16F10 tumors at day 7 after implantation into Rhoj^{GFP/±} mice, and in those of spontaneous breast cancer of 12 weeks old P/Rhoj^{GFP/+} mice. Each indicated regions (squares) are magnified in lower panel. (B) Magnified image showing localization of RhoJ expression in LLC tumor at day 7. Note that expression of RhoJ is mainly confined to tumor ECs (yellow arrowheads), while perivascular mural cells (white arrowheads) and tumor stromal cells (white arrows) also occasionally express RhoJ. (C) Comparisons of RhoJ mRNA expressions in the CD31⁺CD45⁻ ECs (tECs), CD31⁻CD45⁺ hematopoietic cells (tHCs), and CD31⁻CD45⁻ other cells (tOCs) of LLC tumor at indicated days. Each group, n=4. *p<0.05 vs. day 7. (D and E) Temporal changes of RhoJ expressions at indicated days of LLC tumor. The RhoJ-GFP⁺ area is presented as a % per CD31⁺ area. *p<0.05 vs. day 7. (F and G) Spatial changes of RhoJ expressions in peri- and intratumoral regions of various tumors at indicated days. The RhoJ-GFP⁺ area is presented as a % per CD31⁺ area. *p<0.05 vs. each peritumoral region. (H) Images showing RhoJ⁺CD144⁺ tumor vessels (arrow) in human colon cancer tissue and RhoTCD144⁺ normal vessels in adjacent normal colon tissue. Scale bars, 20 μm. (I-K) From TCGA database, total of 216 colon cancer patients were divided into RhoJ-high (n=78) or RhoJ-low (n=138) groups in which the cut-off value was the average RhoJ expression level of all patients. (I) Prevalence of lymphovascular invasion in the colon cancer patients with high or low expression of RhoJ. p=0.017. (J) Kaplan-Meier survival analysis of the colon cancer patients with high or low expression of RhoJ. p=0.033. (K) Correlation plot between RhoJ expression level and the number of metastatic LNs in the colon cancer patients. p=0.006 and R²=0.196. See also FIG. 9 and Table 1.

FIG. 2. RhoJ Deletion Inhibits Tumor Growth, Neovessel Formation, and Metastasis in LLC Tumor. Three weeks after implantation of LLC cells into RhoJ-WT and -KO mice, histological analyses were performed. Unless otherwise denoted: Scale bars, 100 μm. Each group, n=6. Values are mean±SD. *p<0.05 versus RhoJ-WT. (A and B) Comparisons of tumor volume (A) and growth rate (B). Each group, n=10. C) Tumor sections stained with H&E. Arrows indicate hemorrhagic lesions. Scale bar, 5 mm. D) Comparison of intratumoral hemorrhagic area. Each group, n=10. (E and F) Images showing CD31⁺ blood vessels, caspase-3⁺ apoptotic cells, Hypoxyprobe-1⁺ hypoxic areas in tumor. Hypoxyprobe-1™ was IP-injected 90 min before tumor sampling. (G and H) Images (G) and quantification (H) of blood vessels in the peri- and intratumoral regions. (I and J) Images (I) and quantification (J) of vascular sprouts (arrows, sprout >10 μm in length) of tumor vessels. Scale bars, 10 μm. (K and L) Images (K) and quantification (L) of cytokeratin⁺ tumor metastasis in the inguinal LNs. The cytokeratin⁺ area was presented as a % per total sectional area. Scale bar, 500 μm. (M) Lung sections stained with H&E. Four regions were viewed under high magnification. Arrows indicate metastatic foci. Scale bar, 5 mm (upper) and 200 μm (lower). (N) Comparison of number of metastatic colonies (>100 μm in diameter) per lung section. See also FIG. 10.

FIG. 3. RhoJ Deletion Disrupts Tumor Vascular Integrity and Function. Two weeks after LLC implantation, tumors of RhoJ-WT and -KO mice were sampled to analyze detailed vascular phenotypes. Unless otherwise denoted: Scale bars, 100 μm. Values are mean±SD. Each group, n=5. *p<0.05. (A and B) Images (A) and quantification (B) of tumor vessels in the intratumoral center. (C and D) Images (C) and quantification (D) of α-SMA⁺ mural cell coverage on tumor vessels. Coverage of α-SMA is presented as a % of length that lies along CD31⁺ vessels. (E and F) Images (E) and quantification (F) of loss of collagen type IV⁺ BM (red) along tumor vessels (blue). Coverage of collagen type IV is presented as a % of length that lies along CD31⁺ vessels. (G and H) Images (G) and quantification (H) of Ter-119⁺ red blood cells (red) extravasated from tumor vessels. Ter-119⁺ area is presented as a % per total sectional area. (I and J) Images (I) and quantification (J) of dextran leakage area (red) from tumor vessels. Dextran was IV-injected 30 min before sacrifice. Dextran⁺ area is presented as a % per total sectional area. (K and L) Images (K) and quantification (L) of lectin-perfused (red) tumor vessels. Lectin was IV-injected 10 min before sacrifice. Lectin⁺ area is presented as a % per CD31⁺ area. (M) Schematic diagram showing the effects of RhoJ deletion on tumor vasculatures. Red arrows indicate the hemorrhage from vessels. EC, endothelial cell; PC, pericyte; BM, basement membrane; RBC, red blood cell. See also FIG. 11.

FIG. 4. RhoJ Deletion Delays Tumor Growth, Neovessel Formation, and Metastasis in Spontaneous Breast Cancer Model. Tumor growth was analyzed weekly in spontaneous mammary tumors of P/RhoJ-WT and -KO starting from 8 weeks after birth. Samples were harvested 18 weeks after birth. Unless otherwise denoted: Scale bars, 100 μm. Each group, n=5. Values are mean±SD. *p<0.05 versus P/RhoJ-WT. (A) Image showing tumor development at 14 weeks after birth. Dotted lines demarcate palpable mammary tumor nodules. (B) Comparison of percentage of mice remaining tumor-free at indicated time. Each group, n=8. (C) Comparison of number of palpable tumor nodules. Each group, n=8. (D) Comparison of volumes of each tumor nodule at 18 weeks after birth. Lines indicate mean and standard deviation. Each group, n=20. (E) Comparison of total tumor burden. Tumor burden was calculated by summating the volume of tumor nodules per mice. Each group, n=8. (F) Tumor sections stained with H&E showing peri- and intratumoral regions. Acinar structures of P/RhoJ-KO are ductal carcinoma in situ (DCIS), which are non-invasive malignant lesions in mammary ducts during early tumorigenesis. Here the boundary (Black line) between DCIS and the surrounding mammary adipose tissue (Adi) is evident. Invasive carcinoma lesions (Ca) that have invaded into the neighboring stroma are also observable beside the DCIS. On the other hand, the tumor of P/RhoJ-WT have already infiltrated the surrounding tissues and formed solid sheets of tumor cells with little or no DCIS remaining. Nec, Necrotic region. Scale bars, 500 μm. (G and H) Images (G) and quantification (H) of blood vessels in the peri- and intratumoral regions. (I) Comparison of vascular sprouts (>10 μm) per mm² in the peri- and intratumoral regions. (J and K) Images (J) and quantification (K) of dextran leakage (red) from tumor vessels. Dextran was IV-injected 30 min before sacrifice. Dextran⁺ area is presented as a % per total sectional area. (L and M) Images (L) and quantification (M) of coverage of PDGFRβ⁺ mural cells on CD31⁺ tumor vessels. Coverage of PDGFRβ is presented as a % of length that lies along CD31⁺ vessels. (N and O) Images (N) and quantification (O) showing loss of collagen type IV⁺ BM along CD31⁺ tumor vessels. Coverage of collagen type IV is presented as a % of length that lies along CD31⁺ vessels. (P) Lung sections stained with H&E. Metastatic regions were viewed under high magnification. Arrows indicate metastatic foci. Scale bars, 5 mm (upper) and 200 μm (lower). (Q) Comparison of number of metastatic colonies (>100 μm in diameter) in the lung sections. Each group, n=8. See also FIG. 12.

FIG. 5. EC-Specific Ablation of RhoJ Suppresses Tumor Angiogenesis and Induces Vascular Disruption. (A-L) Histological and functional analyses were performed 16 days after implantation of LLC cells into RhoJ-WT^{EC} and -KO^{EC}. Mice were treated with IP injections of tamoxifen (4 mg/kg) 4 times every 2 days starting from the day before tumor implantation. Unless otherwise denoted: Scale bars, 100 μm. Each group, n=5. Values are mean±SD. *p<0.05 versus RhoJ-WT^{EC}. (A) Comparisons of LLC tumor growth. (B) Images of tumor sections stained with H&E. Dotted lines demarcate intratumoral hemorrhagic area. Scale bar, 5 mm (C) Comparison of intratumoral hemorrhage area. (D) Comparison of viable area in cross-sections. (E and F) Images (E) and quantification (F) of CD31⁺ blood vessels in the peri- and intratumoral regions. Dotted lines indicate boundaries between the skin and tumor. (G and H) Images (G) and quantification (H) of coverage of PDGFRβ⁺ mural cells along CD31⁺ tumor vessels. Coverage of PDGFRβ is presented as a % of length that lies along CD31⁺ vessels. (I and J) Images (I) and quantification (J) of loss of collagen type IV⁺ BM along CD31⁺ tumor vessels. Coverage of collagen type IV is presented as a % of length that lies along CD31⁺ vessels. (K and L) Images (K) and quantification (L) of dextran leakage area (green) from tumor vessels. Dextran was IV-injected 30 min before sacrifice. Dextran⁺ area is presented as a % per total sectional area. (M and N) RhoJ-WT^{EC} and -KO^{EC} were treated with IP injections of tamoxifen (4 mg/kg) 4 times on the indicated days (arrows), after the tumor volume had exceeded 300 mm³. Each group, n=10. (M) Comparison of LLC tumor growth. *p<0.05 vs. RhoJ-WT^{EC}. (N) Comparison of overall survival after tamoxifen injection. p=0.016 by log-rank. See also FIG. 13.

FIG. 6. RhoJ Regulates EC Motility, Tube Formation, and Integrity through Suppression of RhoA-ROCK Signaling Pathway. HUVECs were transfected with either control siRNA (siC-ECs) or RhoJ siRNA (siJ-ECs). Unless otherwise denoted: Scale bars, 100 μm. Each group, n=5. Values are mean±SD. *p<0.05 vs. siC-ECs. (A and B) Random migration of ECs was tracked with time-lapse microscopy for 6 hr. (A) Trajectory images showing locomotion of individual ECs. (B) Comparisons of EC migratory speed. (C and D) siC-ECs and siJ-ECs were seeded into the 3D microfluidics system, in which ECs migrate and sprout along growth factor gradient for 3 days. (C) Images showing directional migration and sprouting of ECs. Solid line, starting point; dotted line, point of maximal migration. (D) Comparisons of maximal distance of EC migration and EC sprouting (>10 μm in length). (E-G) siC-ECs and siJ-ECs were seeded on Matrigel and incubated for 12 hr. (E) Images showing EC tube formation. (F) Comparisons of number of EC junctions and tubules. (G) Images showing F-actin fibers (red) in EC tubules. Arrows indicate collapse of ECs and increased actin stress fiber. Indicated region (square) is magnified in the right panel. (H) Images showing F-actin fiber in LLC tumor 16 days after tumor implantation into RhoJ-WT^{EC} and -KO^{EC}. Arrows indicate increased actin stress fiber in tumor vessels. Indicated region (square) is magnified in the right panel. (I and J) siC-ECs or siJ-ECs were cultured on cell inserts until an EC monolayer formed. Subsequently, amount of dextran permeated across the monolayer with or without VEGF-A (50 ng/ml) was measured. (I) Schematic diagram showing in vitro permeability assay. (J) Comparison of vascular permeability across EC monolayer. *p<0.05 vs. siC-EC+PBS. (K and L) siC-ECs or siJ-ECs were cultured on culture plates until an EC monolayer formed. Consecutively, the ECs were incubated with or without VEGF-A (50 ng/ml) for 1 hr. (K) CD144 junctions of EC monolayer in various conditions. (L) Electron microscopic images of EC monolayer in various conditions. Arrows indicate spatial gaps between adjacent ECs. Scale bars, 5 μm. (M) Immunoblotting showing modulation of RhoA-ROCK signaling pathway by RhoJ. siC-ECs or siJ-ECs were cultured for 24 hr, and treated with or without Y-27632 (20 μM) for 1 hr. Three independent experiments show similar results. (N) Schematic diagram showing the role of endothelial RhoJ. When RhoJ is activated in ECs, RhoJ suppresses RhoA-ROCK signaling, while activating N-WASP and PAK, which reorganize the cortical actin filaments in ECs. Upon RhoJ knockdown, RhoA-ROCK signaling is no longer suppressed in ECs, therefore inducing EC contraction through increased formation of actin stress fibers, eventually causing vascular shutdown. N-WASP, Neural Wiskott-Aldrich syndrome protein; PAK, p21-activated kinase. See also FIG. 14.

FIG. 7. Dual Blockade of RhoJ and VEGF Signaling Suppresses Tumor Progression and Metastasis. (A-C) LLC implanted RhoJ-WT or -KO mice were given injections of VEGF-trap (VT) or Fc on the indicated days (arrows). Tumors were sampled 9 days after the first treatment. Scale bars, 100 μm. Each group, n=5. Values are mean±SD. *p<0.05 vs. WT, Fc; ^{#}p<0.05 vs. WT, VT. (A) Comparison of tumor growth. (B and C) Images (B) and quantification (C) of tumor vessels in peri- and intratumoral areas. Dotted lines indicate the boundaries between the skin and tumor. (D-J) LLC implanted WT mice were given injection of either VT or Fc together with either en-siC or en-siJ on the indicated days (arrows). Tissue samples were harvested 12 days after the first treatment. Scale bars, 100 μm. Each group, n=5. *p<0.05 vs. en-siC+Fc; ^{#}p<0.05 vs. en-siC+VT. (D) Comparison of tumor growth. (E and F) Images (E) and quantification (F) of tumor vessels in peri- and intratumoral regions. (G and H) Images (G) and quantification (H) of intratumoral hemorrhage. ^{$}p<0.05 vs. en-siJ+Fc. Scale bar, 5 mm. (I and J) Images (I) and quantification (J) of metastasized cytokeratin⁺ tumor cells (red) to the inguinal LNs. Cytokeratin⁺ area is presented as % per total sectional area. Scale bar, 500 μm. See also FIG. 15.

FIG. 8. RhoJ Blockade Augments the Anti-Tumor Effect of a VDA, Combretastatin-A4-Phosphate (CA4P). Unless otherwise denoted: Scale bars, 100 μm. Values are mean±SD. (A and B) siC-ECs and siJ-ECs were seeded on Matrigel with or without CA4P (20 nM) and incubated for 12 hr. *p<0.05 vs. siC-EC+PBS; ^{#}p<0.05 vs. siC-EC+CA4P. (C) siC-ECs and siJ-ECs were cultured on cell inserts until an EC monolayer formed. The results of dextran permeation across the EC monolayer for 20 min was compared. *p<0.05 vs. siC-EC+PBS; ^{#}p<0.05 vs. siC-EC+CA4P. (D-H) LLC implanted RhoJ-WT or -KO mice were given IP injections of CA4P (50 mg/kg) or PBS on the indicated days (arrows). Tissues were sampled 6 days after the first treatment. Each group, n=5. *p<0.05 vs. WT, PBS; ^{#}p<0.05 vs. WT, CA4P. (D) Comparison of tumor growth. ^{$}p<0.05 vs. KO, PBS. (E and F) Images (E) and quantification (F) of tumor vessels in the peri- and intratumoral regions. (G and H) Images (G) and quantification (H) of metastasized cytokeratin⁺ tumor cells (red) to inguinal LNs. Cytokeratin⁺ area is presented as % per total sectional area. Scale bar, 500 μm. See also FIG. 16.

FIG. 9. The Expression of RhoJ in Tumor Tissue and Normal Organs. Unless otherwise denoted: Scale bars, 100 μm. Each group, n=5. Values are mean±SD. (A) Schematic representation of the Rhoj targeted allele. The genomic structure after neo-cassette removal is shown on the third line. The genomic structure after loxP-cassette removal is shown on the fourth line. (B) Genotypes of Rhoj-flox allele and Rhoj-KO allele. (C) The lack of RhoJ expression in RhoJ-KO mice was confirmed by RT-PCR. (D-E) CD31⁻CD45⁻ cells were purified using FACS from the spontaneous mammary tumors of 12 weeks old P/Rhoj^{GFP/+} mice. The mRNA levels of various genes in GFP⁺ and GFP⁻ cells are compared. (D) Purification of GFP⁺ and GFP⁻ cells from CD31⁻ CD45⁻ cells in tumors of the P/Rhoj^{GFP/+} mice. (E) Comparisons of mRNA levels of various genes related to stromal cells between the GFP⁺ and GFP⁻ cells. Each group, n=3. *p<0.05 versus GFP⁺ cells (F) Comparison of the double CD31⁺ and GFP⁺ expressing area in total GFP⁺ area. The double CD31⁺ and GFP⁺ area was presented as a % per total GFP⁺ area. (G) Organs from 8-weeks old Rhoj^{GFP/+} mice were sampled and analyzed to detect RhoJ expression. Images show RhoJ expression (green) in heart, lung, lymph node, kidney, liver, and spleen of adult mice. Arrowheads indicate high expression of RhoJ in CD31⁺ blood vessels. (H-O) Indicated organs of 8-weeks old RhoJ-WT and -KO mice were sampled and sectioned for morphological analyses. Note that there are no differences in vascular morphology, density, and integrity between RhoJ-WT and RhoJ-KO mice even at ultra-structural level. (H) Images of the indicated organs stained with H&E. (1-N) Images and quantification of CD31⁺ blood vessels (blue) and CD144⁺ EC junctions (red) in the endocardial (Endo), myocardial (Myo), and pericardial (Peri) regions of the hearts, lungs, livers, and kidneys. CD144⁺ area/CD31⁺ area indicate the percentage of CD144⁺ area per the total CD31⁺ blood vessel area. CV, central vein; GM, glomerulus. Each group, n=3. (O) Electron microscopic images of blood vessels and their surroundings in indicated organs. EC, endothelial cell; CM, cardiomyocyte; R, red blood cell; Alv, alveolar space; Pod, podocyte. Scale bars, 2 μm. This Figure is related to FIG. 1.

FIG. 10. RhoJ Deletion Did Not Affect Lymphangiogenesis in LLC Tumor. (A and B) Three weeks after implantation of LLC cells into RhoJ-WT and -KO mice, histological analyses of tumors and inguinal lymph nodes were performed. Images (A) and quantifications (B) of LYVE-1⁺ lymphatic vessels in the peritumoral regions of implanted tumors and inguinal lymph nodes. White dotted lines indicate the boundaries between the skin and tumor. White solid lines indicate the margin of lymph nodes. Scale bars, 100 μm. Each group, n=5. Values are mean±SD. This Figure is related to FIG. 2.

FIG. 11. RhoJ Deletion Suppresses B16F10 Melanoma Tumor Growth and Progression. Seventeen days after B16F10 melanoma implantation, tumors and inguinal LNs of RhoJ-WT and -KO mice were sampled. Unless otherwise denoted: Scale bars, 500 μm. Each group, n=6. Values are mean±SD. *p<0.05 versus RhoJ-WT. (A) Comparison of melanoma tumor growth. (B) Tumor sections stained with H&E. Scale bar, 5 mm. (C) Low magnification Images showing CD31⁺ blood vessels in melanoma tissue. White solid lines indicate the outer margin of the skin, while white dotted lines indicate the boundaries between the skin and tumor. (D) Comparisons of blood vessel densities in peri- and intratumoral regions. (E) Images showing melan-A⁺ tumor metastasis in the inguinal LNs. (F) Comparison of melanoma tumor metastasis in the inguinal LNs. The melan-A⁺ area is presented as a % per total sectional area. This Figure is related to FIG. 3.

FIG. 12. RhoJ Deletion Suppresses Wound Healing. Round 5-mm full thickness wounds were made on the dorsal skin of 8-weeks old RhoJ-WT and -KO mice using a biopsy punch. The progression of wound healing was observed and photographed over the following 6 days. Unless otherwise denoted: Scale bars, 500 μm. Each group, n=5. Values are mean±SD. *p<0.05 vs. RhoJ-WT. (A) Images showing RhoJ-GFP expressions (green) in CD31⁺ blood vessels (red) of the granulation area two days after the punch wound. Dotted line indicates the margin of granulation tissue. Scale bar, 50 (B) Gross pictures showing wound healing process after the punch wound. (C) Quantification of remaining wound area, defined by relative wound size at each time points compared to the initial wound size. (D) Images of wound sections stained with H&E. RhoJ-KO mice showed delayed epidermal healing of wound lesion with reduced granulation tissue (Gr). Dotted lines indicate the margin of epidermis (Epi), which are covered by crust (Cr). (E) Images showing the CD31⁺ blood vessels (red) in wound lesion. White solid lines indicate the outer margin of the skin, while white dotted lines indicate the margin of granulation tissue (Gr). (F and G) Quantifications of vascular densities (F) and granulation areas (G) in the wound lesions. This Figure is related to FIG. 4.

FIG. 13. Effective and Selective Knockdown of RhoJ in ECs of Lung and Tumor, and Distinct Features of Tumor Vessels in RhoJ-KO^{EC} Mice. (A-C) Cdh5(PAC)-CreER^{T2};Rhoj^{fl/fl} mice (RhoJ-KO^{EC}) and Rhoj^{fl/fl} mice (RhoJ-WT^{EC}) were treated with intraperitoneal injections of tamoxifen (4 mg/kg) 4 times every 2 days starting from the day before tumor implantation. CD31⁺CD45⁻ ECs and CD31⁻CD45⁻ other cells (OCs) in tumor and lung tissue were purified from RhoJ-WT^{EC} and RhoJ-KO^{EC} mice. Unless otherwise denoted: Each group, n=3. Values are mean±SD. *p<0.05 vs. RhoJ-WT^{EC}. (A) Images showing the effective excision of RhoJ allele in the lungs of RhoJ-KO^{EC} mice treated with 4 times injections of tamoxifen. (B) Effective (˜98%) and selective knockdown of RhoJ in the purified lung ECs of RhoJ-KO^{EC} mice after tamoxifen injection. (C) Effective (˜70%) and selective knockdown of RhoJ in the purified tumor ECs of RhoJ-KO mice after tamoxifen injection. (D) High magnification images of tumor vessel in intratumoral center. The tumor vessels of RhoJ-WT^{EC} mice display several vascular sprouts (arrows), whereas RhoJ-KO^{EC} mice have more disintegrated and fragmented tumor vessels (arrowheads) without distinct vascular sprouts. Scale bars, 50 (E) Images showing CD144⁺ EC junctions in tumor vessels of intratumoral region. Three independent experiments show similar results. Scale bars, 100 μm. This Figure relates to FIG. 5.

FIG. 14. The Roles of RhoJ in Primary Cultured ECs and Tumor Vasculatures. (A-C, F-M) HUVECs were transfected with either control siRNA (siC) or 5 independent RhoJ siRNA (J0, J1, J2, J3, and J4). Among 5 RhoJ siRNAs, 3 (J0, J1, and J2) were chosen for further evaluation. Cells were incubated for 24 hr. Unless otherwise denoted: Each group, n=3. Values are mean±SD. *p<0.05 vs. siC. (A) Quantitative PCR showing the efficiency of each siRNA in knockdown of RhoJ in HUVECs. (B) Immunoblotting showing the effective knockdown of RhoJ in HUVECs. (C) Comparisons of EC migratory speed. Random migration of HUVECs was tracked with time-lapse microscopy for 6 hr. (D) Schematic diagram showing 3D microfluidics system for angiogenesis assay. The microfluidics system consists of 3 channels. Central channel (blue) is filled with fibrin gel, which is 500 μm wide and 100 μm high, and serves as a scaffold for the EC migration and sprouting. HUVECs were seeded in the left channel (purple) with media. Lung fibroblasts were seeded with fibrin gel in the right channel (green). HUVECs migrated and sprouted along the concentration gradient from left to right channel, finally resulting in formation of an EC network 3 days later. (E) Image shows EC network, which was formed on fibrin scaffold of the central channel of 3D microfluidics system. Scale bar, 100 μm. (F) Comparisons of the number of EC junctions and tubules. HUVECs were seeded on Matrigel and incubated for 12 hr. (G) Images showing F-actin fibers (green) in the HUVECs at 24 hr after the transfection. Note that there are increased F-actin stress fibers (arrow) in the HUVECs transfected with J0. Scale bars, 10 μm. (H) Comparison of vascular permeability across EC monolayer. The HUVECs were cultured on collagen-coated 1.0 μm-size pore insert until an EC monolayer formed. After starvation for 12 hr, the cells were treated with or without VEGF (50 ng/ml) for 2 hr and was then incubated with 70 kDa FITC-Dextran for 20 min. Subsequently, amount of dextran permeated across the monolayer was measured. (I) Electron microscopic images and outline drawings of tumor vessels in the intratumoral region of LLC tumor. The tumor blood vessels of RhoJ-KO mice show more severely disrupted and malformed morphology with almost all or complete loss of endothelial lining (arrows) compared to RhoJ-WT mice. T, tumor cell; EC, endothelial cell; Lu, lumen; W, white blood cell. Scale bars, 5 μm. (J-M) HUVECs were cultured at 40% confluence, starved overnight, treated with VEGF-A (50 ng/ml) for 10 min, and lysed. The RhoA activities were determined using the rhotekin-bead to pull down the active GTP-bound RhoA within cell lysates and by performing Immunoblotting with anti-RhoA antibody. ROCK activities were determined by incubating cell lysate in plates pre-coated with recombinant MYPT1, from which MYPT1 is phosphorylated by active ROCK and detected with anti-phospho-MYPT1 antibody. (J) Immunoblotting showing increased activation of RhoA and increased phosphorylation of MLC in three different RhoJ-knockdown HUVECs. Three independent experiments showed similar results. (K) Comparisons of RhoA activities and MLC phosphorylations. (L) Comparisons of ROCK activities. Relative absorbance of each sample at 450 nm was compared. (M) Comparisons of ROCK activities after treatment with various concentrations (0, 1, 2, 5, 10, and 20 μM) of Y-27632 in HUVECs transfected with siC and J0. The cells were incubated with medium containing indicated concentrations of Y-27632 for 1 hr. This Figure is related to FIG. 6.

FIG. 15. Enhanced Effect of Cisplatin in RhoJ-KO and Application of Tumor Targeted siRNA Delivery System Using EDB-Aptide Conjugated Liposome. (A-E) LLC implanted RhoJ-WT or -KO mice were given injections of cisplatin (10 mg/kg) or PBS on the indicated days (arrows). Tissues were sampled 2 weeks after cisplatin treatment. Unless otherwise denoted: Each group, n=5. Values are mean±SD. *p<0.05 vs. RhoJ-WT; ^{#}p<0.05 versus RhoJ-WT, Cisplatin. (A) Quantification of the tumor growth. (B) Tumor sections stained with H&E. Dotted lines demarcate the necrotic regions. Scale bar, 5 mm. (C) Quantification of the necrotic area per sectional area. (D) Images showing cisplatin-modified DNA (red) in the intratumoral region of LLC tumor treated with cisplatin. Scale bars, 100 μm. (E) Quantification of intratumoral cisplatin-modified DNA area. The area is presented as % per total sectional area. (F) Structure of the aptide consisting of a scaffold region and a target binding region. (G) Structure of APT_{EDB} conjugated liposome (APT_{EDB}-liposome) complex, in which the siRNA is loaded inside the APT_{EDB}-liposome. (H) Images showing the targeted delivery of APT_{EDB}-liposome into LLC tumor. APT_{EDB}-liposome or APT_{scr}-liposome (negative control) was labeled with Cy5.5 and IV injected into LLC tumor-bearing mice. Near-infrared images are taken at the indicated time points using an IVIS imaging machine. Tissues were sampled 4 hr after the APT-liposome injection and analyzed. Note the enhanced uptake of liposome complexes in the LLC tumor tissue of APT_{EDB}-liposome injected mice (yellow arrowhead). Red color denotes high liposome complexes uptake, while blue color denotes low uptake. (I) Quantitative PCR result showing the minimal expression of RhoJ in cultured LLC tumor cells compared to murine endothelial cells (MS1). Each group, n=3. Values are mean±SD. *p<0.05 versus MS1. (J) Quantitative PCR result showing the minimal expression of RhoJ in purified CD31⁻CD45⁻ tumor other cells (tOCs) compared to purified CD31⁺CD45⁻ tumor ECs (tECs). Each group, n=3. Values are mean±SD. *p<0.05 versus tEC. (K) Immunoblotting showing the effective knockdown of RhoJ in the LLC tumor tissues of en-siC or en-siJ treated mice. Tumor tissues were harvested after five IV injections of siRNA which was encapsulated in APT_{EDB}-LS. This Figure is related to FIG. 7.

FIG. 16. Strengthened Actions of CA4P on MLC Phosphorylation by RhoJ Blockade. HUVECs were transfected with either control siRNA (siC-EC) or RhoJ siRNA (siJ-EC) and treated with either CA4P (20 nM) or PBS for 30 min. Immunoblotting showing the phosphorylation of MLC. MLC phosphorylation is further increased when siJ-ECs are treated with CA4P. Three independent experiments show similar results. This Figure is related to FIG. 8.

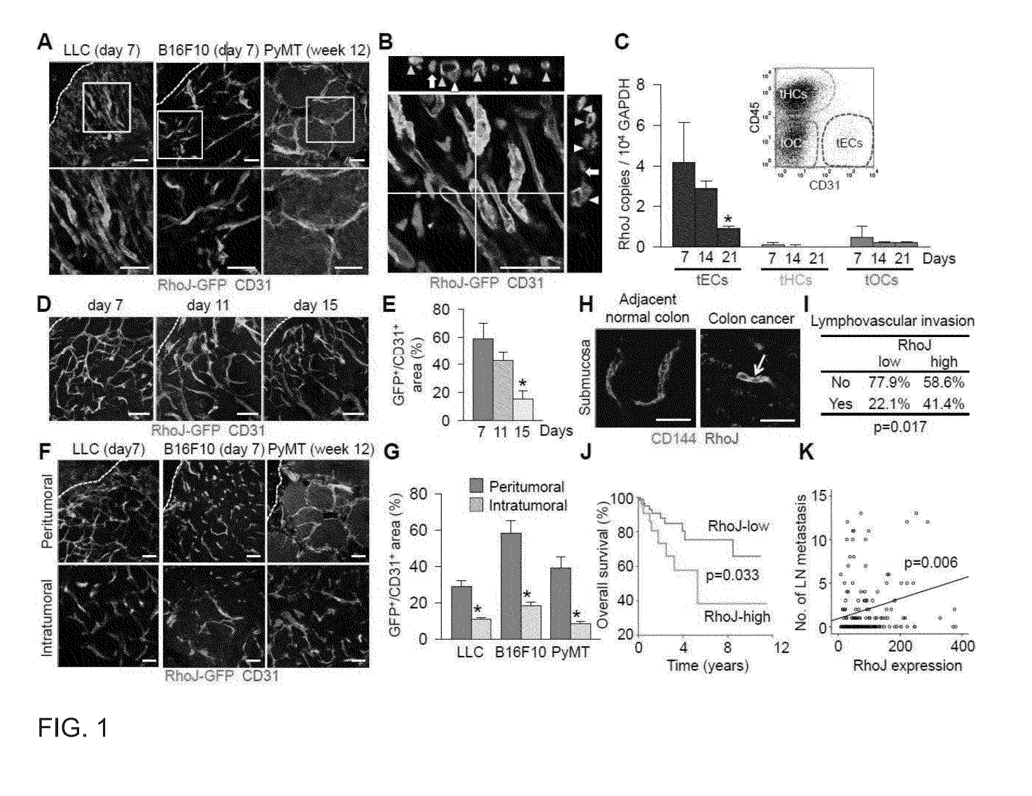

Table 1 shows characteristics of the patient cohort (n=216) as related to FIG. 1.

### DETAILED DESCRIPTION OF THE PREFERRED EMBODIMENTS

In the present application, “a” and “an” are used to refer to both single and a plurality of objects.

The term “about” as used herein refers to the usual error range for the respective value readily known to the skilled person in this technical field.

As used herein, “RhoJ blockade” or “inhibiting RhoJ activity” refers to the inhibition of RhoJ activity in an organism. The inhibition may occur by binding RhoJ with selective small molecules that nullify the RhoJ activity, or depleting RhoJ at the protein level with an anti-sense oligonucleotide such as an siRNA, or inhibiting a member in the RhoJ reaction cascades that results in reduction of RhoJ activity.

As used herein, “a chemical compound that inhibits the activity of RhoJ” refers to a chemotherapeutic agent that is specific to RhoJ. Such compounds may be produced by synthesizing chemicals and assaying for their activity against RhoJ activity.

As used herein, “disrupting tumor vascular integrity” refers to specifically and selectively destroying the blood vessels present in a tumor, but not normal blood vessels as is manifest as intratumoral hemorrhagic necrosis.

As used herein, “sequestering VEGF” refers to a molecule such as VEGF-trap, which is used to bind VEGF so that VEGF is neutralized and is not active.

As used herein, “vascular-disrupting agent” refers to an agent known in the art that disrupts pre-formed blood vessels, such as combretastatin-A4-phosphate.

As used herein, “cytotoxic therapeutic agent” refers to any agent that is toxic to a cell, and includes a substance that inhibits or prevents the function of cells and/or causes destruction of cells. The term includes radioactive isotopes, toxins such as small-molecule toxins or enzymatically active toxins of bacterial, fungal, plant or animal origin, or fragments thereof. Preferably, the agent is a chemotherapy drug, such as cisplatin.

Known anti-cancer chemical compounds that are useful in the treatment of cancer exist, and may be used together with the inventive anti-RhoJ compounds. Examples of some of these compounds include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®)), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal1 and calicheamicin omegaI1 (see, e.g., Nicolaou et al., Angew. Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

As used herein, administration “in combination with” one or more further therapeutic agents includes simultaneous (concurrent) and consecutive administration in any order.

As used herein, “antagonist” refers to a ligand that tends to nullify the action of another molecule.

As used herein, “carriers” include pharmaceutically acceptable carriers, excipients, or stabilizers which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Carrier as used in the present application includes “aptide”, which refer to a class of high-affinity peptides, which are typically conjugated to another entity such as drug-containing liposomes for cancer therapy. Such drug may include an oligonucleotide such as siRNA or a protein or a chemical compound.

As used herein, “consisting essentially of” when used in the context of a nucleic acid sequence or amino acid sequence refers to the sequence that is essential to carry out the intended function of the amino acid encoded by the nucleic acid.

As used herein, “effective amount” is an amount sufficient to effect beneficial or desired clinical or biochemical results. An effective amount can be administered one or more times. For purposes of this invention, an effective amount of an inhibitor compound is an amount that is sufficient to palliate, ameliorate, stabilize, reverse, slow or delay the progression of the disease state.

As used herein, “ligand” refers to any molecule or agent, or compound that specifically binds covalently or transiently to a molecule such as a polypeptide. When used in certain context, ligand may include antibody. In other context, “ligand” may refer to a molecule sought to be bound by another molecule with high affinity, such as in a ligand trap.

As used herein, “mammal” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

As used herein, “subject” is a vertebrate, preferably a mammal, more preferably a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-structures, such as polyamides.

Role of RhoJ in the Regulation of Tumor Angiogenesis and Tumor Vascular Integrity

Here, we have demonstrated a critical role of RhoJ in the regulation of tumor angiogenesis and tumor vascular integrity. The phenotypic endpoints of RhoJ blockade are similar to those of blockade of another small GTPase, R-Ras; genetic disruption of R-Ras also severely impairs EC barrier function, resulting in disturbed tumor vasculature maturation (Sawada et al., 2012). Furthermore, it has recently been reported that RhoJ is strongly expressed in human cancers, being one of the top 10 genes of the common angiogenesis signature (Masiero et al., 2013). This correlates well with our data showing that high expression of RhoJ in colon cancer is a negative prognostic factor in these patients, further highlighting RhoJ as a clinically relevant therapeutic target in cancer.

RhoJ blockade displayed several advantages over current vascular targeting therapy, but the most superior advantage is its “double assault” on tumor vessels. Vascular targeting agents developed during the past decade are commonly classified as either AIAs or VDAs. AIAs mainly suppress the formation of tumor neovessels and induce tumor vessel normalization, whereas VDAs directly disrupt preformed tumor vessels and shut down blood flow, finally resulting in massive tumor necrosis and hemorrhage (Tozer et al., 2005). AIAs are particularly effective in the peritumoral regions of newly progressing tumors where new tumor vessels are robustly developing, while VDAs are most effective in the intratumoral regions of established tumor where preformed immature vessels are abundant (Horsman and Siemann, 2006; Siemann, 2011). RhoJ blockade encompasses both aspects of AIAs and VDAs and offers an effective strategy for targeting tumor vasculatures: It simultaneously impedes the formation of tumor neovessel and disrupts the pre-established tumor vessel network. Through this “double assault” on tumor vasculature, RhoJ blockade markedly inhibited blood flow to tumor cells and displayed a convincing anti-cancer and anti-metastatic effect.

In addition, RhoJ blockade compensates for and augments other anti-cancer therapies. The combination therapy of RhoJ blockade and the conventional chemotherapeutic drug, cisplatin, proved to be very effective in delaying tumor progression. As is previously known, the intratumoral core of tumors is resistant to conventional anti-cancer therapies (Trédan et al., 2007; Wachsberger et al., 2003), because anti-cancer drug delivery to this core is limited and inefficient due to the immature tumor vessels and increased interstitial pressure (Fukumura and Jain, 2007). Additionally, tumor cells in the intratumoral core have an intrinsic resistance to chemotherapy because they proliferate slowly and the growth fraction is small (Trédan et al., 2007). Intriguingly, RhoJ blockade preferentially induces vascular shutdown in intratumoral regions, resulting in necrosis of the tumor cells. By combining cisplatin and the RhoJ blockade, both of which exert distinctive modes of action, we achieved a comparatively enhanced anti-tumor and anti-metastatic effect, which suggests the potential of RhoJ blockade as an adjuvant for conventional chemotherapies. Moreover, the combination of RhoJ blockade with VDAs also showed an enhanced anti-tumor efficacy. Most VDAs target the tubulin cytoskeleton of tumor ECs directly and induce activation of RhoA-ROCK signaling in tumor ECs, resulting in the rapid and selective disruption of the preformed tumor vessels (Siemann, 2011). However, in spite of promising preclinical results, they failed to show efficacy in clinical trials (Baguley and McKeage, 2012). The major drawback of VDAs is that they mainly target the intratumoral core, leaving the remaining peripheral viable rim to regrow and even acquire resistance to VDAs (Horsman and Siemann, 2006; Tozer et al., 2005). In contrast, RhoJ blockade in the present study exerted its anti-tumor effect through inhibition of neovessel formation in both the peri- and intratumoral regions and also enhanced shutdown of pre-existing tumor vessels in the intratumoral regions. Furthermore, we found that RhoJ blockade shares its action mechanism with VDAs, also activating the RhoA-ROCK signaling pathway. In this regard, it is logical to speculate that RhoJ blockade may be complementary to current VDA therapies. Indeed, we confirmed that RhoJ blockade could overcome the resistance acquired from VDA monotherapies, such as CA4P, with regard to tumor growth and progression.

Previous studies have found that VEGF-A stimulation regulates the activity of various Rho GTPases, such Cdc42, Rac 1, and RhoA, whereas interactions among various Rho GTPases are poorly understood (Beckers et al., 2010; Bryan and d'Amore, 2007; Schiller, 2006). Blocking the RhoJ pathway over a prolonged period raises the possibility of compensatory activation of other Rho GTPases in tumor vessels, especially by Cdc42 and Racl, which share common downstream effector molecules with RhoJ (Leszczynska et al., 2011). From this perspective, the concurrent inhibition of RhoJ signaling and VEGF-A signaling could be an attractive therapeutic strategy not only by enhancing current AIA therapy but also by maximizing the vascular-disrupting effect of the RhoJ blockade. Indeed, our findings strongly support this possibility. The combination of RhoJ blockade and VEGF decoy receptor, VEGF-trap, showed comparatively potent anti-angiogenic activity in both peri- and intratumoral areas of the LLC tumor, which are known to be resistant to conventional AIA therapies (Shojaei et al., 2007). Another possible benefit from this combination is that RhoJ blockade may maintain and maximize responses to the AIA therapies. It is known that tumor vessels regrow alongside the ghost tracks of remnant BM after cessation or during the resting period of AIA treatment (Mancuso et al., 2006). Intriguingly, we observed a severe loss of BM in RhoJ-deficient tumor vessels, indicating that concurrent RhoJ blockade might abolish remnant BM in concert with AIAs and prevent tumor vessel from regrowth, finally resulting in a sustained response to the AIA therapies.

An additional advantage of RhoJ blockade is that it selectively targets tumor vessels with minimal systemic side effects. Current AIAs influence normal vessels as well because their main targets, VEGF-A and its receptors, are expressed ubiquitously. Therefore, they induce systemic side effects such as hemorrhage, hypertension, proteinuria, and delayed wound healing (Chen and Cleck, 2009; Kamba and McDonald, 2007). On the other hand, RhoJ expression is very specific to pathologic conditions, especially in tumor tissues, while being rarely expressed in organs under normal physiologic conditions; the global deletion of RhoJ does not induce gross abnormalities and lethality. However, our results indicate that RhoJ plays a positive angiogenic role during wound healing, and this could be an unavoidable side-effect of a putative RhoJ inhibitor.

Finally, RhoJ is a feasible target for clinical drug development. We could therapeutically target RhoJ in tumor tissues through an in vivo siRNA delivery system. Using the APT_{EDB}-LS complex as a carrier, which has high specificity against tumor tissues (Kim et al., 2012), we effectively delivered siRhoJ into tumor tissues and significantly delayed tumor growth and metastasis, especially in concert with VEGF-trap. Consequently, we established a way to clinically inhibit RhoJ.

In conclusion, our evidence shows that RhoJ is a promising selective target in the tumor vasculature that governs the processes of tumor angiogenesis and vascular integrity. The distinguishing characteristics of RhoJ blockade provide a strategy for overcoming the limitations of current vascular targeting therapies in patients with advanced cancer. Further development of specific RhoJ inhibitors is needed to ascertain their efficacy and safety in clinical settings.

### Therapeutic Composition

In one embodiment, the present invention relates to anti-cancer treatment

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitised in vitro and adoptively transferred to the recipient). Depending on the route of administration, the peptide may be required to be coated in a material to protect it from the action of enzymes, acids and other natural conditions which may inactivate said ingredients.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides or chemicals or oligonucleotides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, thus requiring only a fraction of the systemic dose.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Experimental Methods

### Example 1.1

### Mice

Animal care and experimental procedures were performed under the approval (KA2011-17) from the Animal Care Committee of KAIST. Specific pathogen-free (SPF) C57BL/6J and MMTV-PyMT transgenic mice (FVB/N) were purchased from Jackson Laboratory (Bar Harbor, Me.). Rhoj^{GFP/GFP} and Rhoffl mice, and Cdh5(PAC)-CreER^{T2} mice (Wang et al., 2010b) were transferred and bred in our SPF facilities. To deplete Rhoj in MMTV-PyMT tumors, Rhoj^{GFP/GFP} female mice were intercrossed with MMTV-PyMT male mice. To deplete Rhoj specifically in ECs, Rhof^{fl/fl} mice were intercrossed with Cdh5(PAC)-CreER^{T2} mice. All mice were fed with ad libitum access to standard diet (PMI Lab diet) and water. All mice were anesthetized by intramuscular injection of a combination of anesthetics (80 mg/kg of ketamine and 12 mg/kg of xylazine) before being sacrificed.

### Example 1.2

### Tumor Models and Treatment Regimens

LLC and B16F10 melanoma cells were obtained from American Type Culture Collection. To generate implantation tumor models, suspensions of tumor cells (1×10⁶ cells in 100 μl) were SC injected into the dorsal flank of 8 to 10 weeks old mice. Tumor volumes were measured at given time points. Tumor volume was calculated according to the formula, 0.5×A×B², where A is the largest diameter of a tumor and B is its perpendicular diameter. Tumor growth rate is defined as increased tumor volume relative to 2 days before. Indicated days later, the mice were anesthetized and tissues were harvested for further analyses. Tamoxifen (4 mg/kg, 4 times every 2 days, Sigma-Aldrich) was IP injected into Cdh5(PAC)-Cre^{ERT2};Rhor^{fl/fl} mice starting from the day before tumor implantation or after the tumor volume had exceeded 300 mm³. Cisplatin (10 mg/kg, every 7 days, Sigma-Aldrich) is IP injected for cytotoxic chemotherapy when tumor volume exceeded 100 mm³. VEGF-trap (25 mg/kg, indicated schedule) is SC injected as an AIA therapy. CA4P (50 mg/kg, every 2 days, Sigma-Aldrich) was IP injected as a VDA therapy. As a control, equal amounts of Fc or PBS was injected in the same manner. To knock down RhoJ in vivo, control or RhoJ siRNA (2 mg/kg, indicated schedule), which were encapsulated into apt_{EDB}-LS complexes, were IV injected into tumor-bearing mice.

### Example 1.3

### Human Tumor Specimens

All human samples were collected by the tissue bank of Severance Hospital, Seoul, Korea, with the informed consents from the donors, following the bioethics and safety regulations. All procedures regarding human samples were performed with the approval of institutional review board (KH2013-02).

### Example 1.4

### Generation of RhoJ-KO Mice

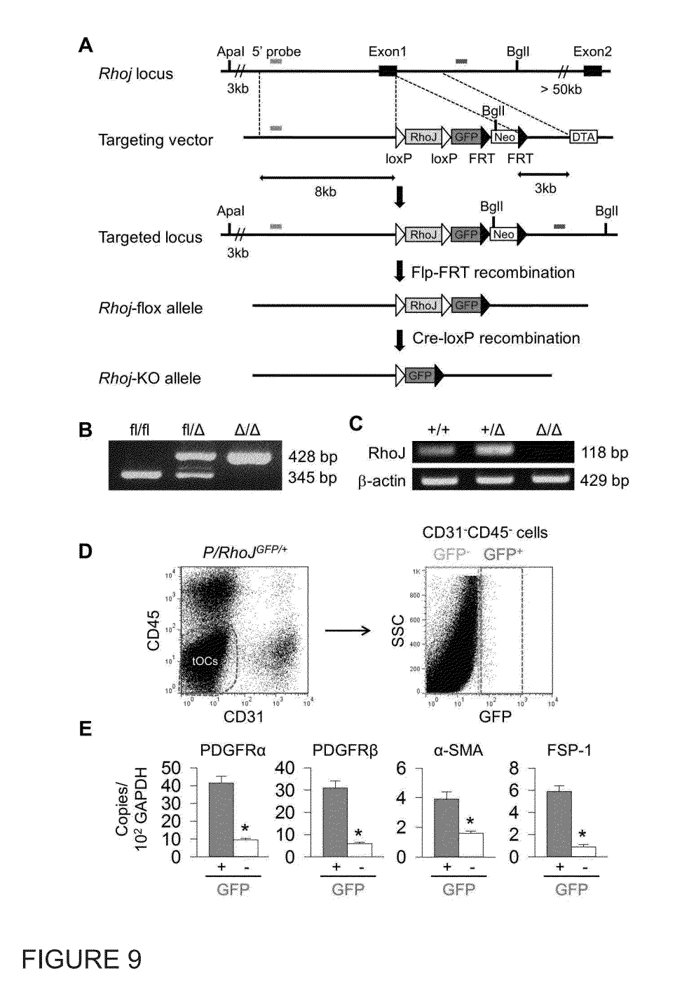

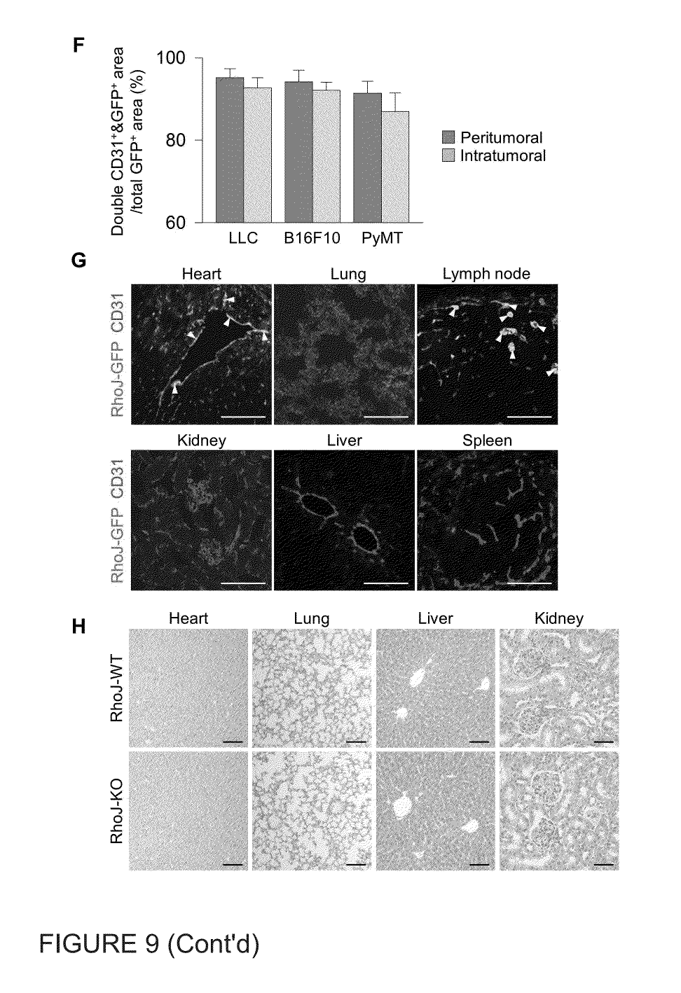

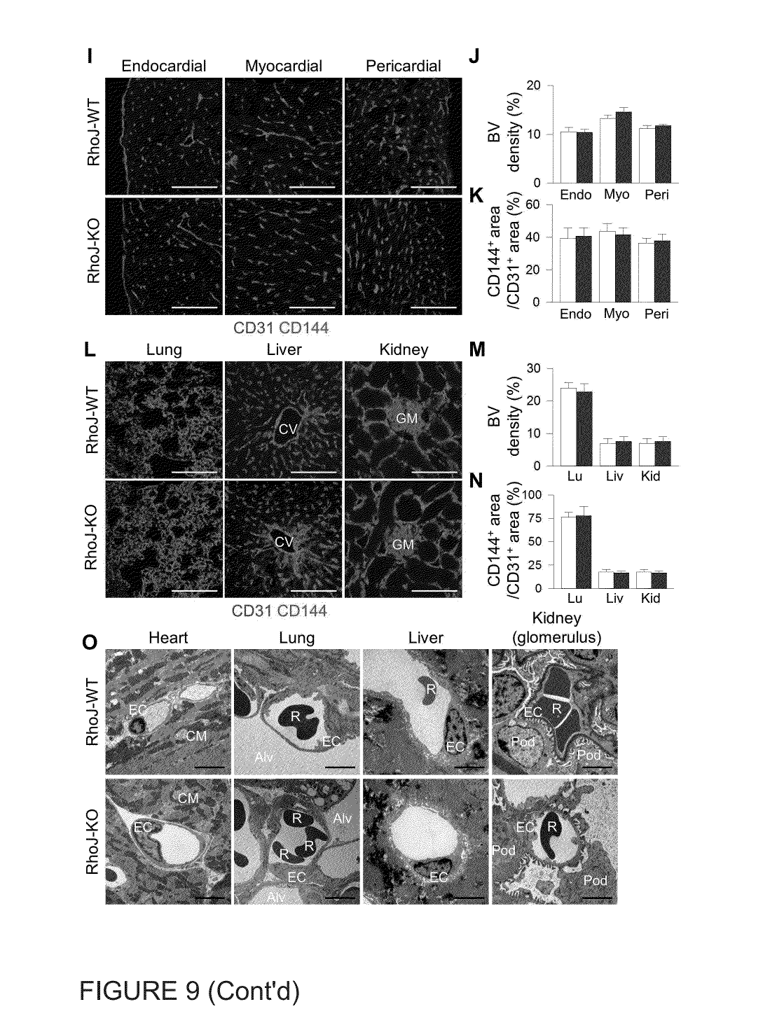

To generate Rhoj mutant mice, a targeting construct was assembled, which contains a loxP-mouse Rhoj cDNA-pA-loxP-EGFP-pA-FRT-SV40 early promoter-Neo-pA-FRT cassette (Uesaka et al., 2007) flanked by 8-kb 5′ and 3-kb 3′ arms which were generated by PCR using a C57BL/6-derived BAC clone RP23-280114 (BACPAC Resource Center) as a template (FIG. 9A). TT2 ES cells (Yagi et al., 1993) were electroporated with the linearized targeting construct, were positively selected with G418, and were confirmed by Southern blotting. Targeted ES cells were injected into ICR 8-cell stage embryos. Resulting mice were sequentially mated with the ACTFLPe transgenic (Tg) (Rodriguez et al., 2000) (Jackson Laboratory) and EIIaCre Tg (Lakso et al., 1996) (Jackson Laboratory) mice to generate Rhoj-flox and Rhoj-KO alleles (FIG. 9A). Rhoj-KO allele expresses GFP under the transcriptional control of the RhoJ gene. The offspring were further backcrossed to C57B1/6 more than 10 times. Mice were genotyped using PCR (FIG. 9B) with following primers: a forward primer 5′-GACCCTTTTCATCCCTCCTC-3′ (SEQ ID NO:1), a reverse WT/flox primer 5′-TCTCCTCATGTCCATTGCAG-3′ (SEQ ID NO:2) (WT, 247 bp; flox, 345 bp), and a reverse KO primer 5′-GAACTTCAGGGTCAGCTTGC-3′ (SEQ ID NO:3) (KO, 428 bp). The absence of the RhoJ mRNA in the RhoJ-KO mice was confirmed by RT-PCR as shown above (FIG. 9C), using a forward primer 5′-GCTACGCCAACGACGCCTTC-3′ (SEQ ID NO:4) (exon 1) and a reverse primer 5′-TGTCCTGCAGTGTCGTATAGTCCA-3′ (SEQ ID NO:5) (exon 2).

### Example 1.5

### Preparations of Reagents

To produce recombinant proteins, dimeric-Fc (Fc) and VEGF-trap, stable CHO cell lines that secrete these recombinant proteins were used as previously described (Koh et al., 2010). Recombinant proteins in supernatant were purified by column chromatography with Protein A agarose gel (Oncogene) using acid elution. After purification, the recombinant proteins were quantified using the Bradford assay and confirmed by Coomassie blue staining after SDS-PAGE.

### Example 1.6

### Skin Wound Healing Model

The skin wound healing assay was conducted as described previously (Zhou et al., 2004). Two round 5-mm full-thickness punch wounds were made on the dorsal skin of 8-week old mice using a biopsy punch (Miltex). The progression of wound healing was observed and photographed every 2 days over the following 6 days. At day 6 after creating the wound, the wound tissue was harvested for histologic analyses.

### Example 1.7

### Bioinformatics Data Set and Analysis

To analyze the influence of RhoJ expression in human cancer patients, we acquired the mRNA sequencing (RNASeq) data from The Cancer Genome Atlas (TCGA) database (http://cancergenome.nih.gov). Of the 438 colon cancer patients registered in TCGA, 405 patients had the RNASeq data available. Among various RNASeq platforms, we chose Illumina HiSeq V2 (RNASeqV2), the platform with which the largest number of patients were included (n=216). MapSplice (Wang et al., 2010a) was used for alignment and RNASeq by Expectation Maximization (RSEM) (Li and Dewey, 2011) was used to determine RhoJ expression levels. Of the 216 colon cancer patients, the RNA SeqV2 level 3 data were used to obtain the normalized RhoJ expression levels and clinical data were used to obtain various clinical attributes which were summarized in Table 1. The survival attribute was computed from ‘days_to_last_followup’ or ‘days_to_last_known_alive’ if the patients are still alive, and ‘days_to_death’ if the patients are dead. The clinical outcome attribute indicates whether the patient is dead (1) or not (0). Each patient in the TCGA database has their own ID so we are able to map every tissue sample to the corresponding patient. All 216 colon cancer patients were divided into RhoJ-high (n=78) or RhoJ-low (n=138) groups in which the cut-off value was the average RhoJ expression level of all patients. Every processing related to the TCGA database was done with Python software.

### Example 1.8

### Histological Analyses

For hematoxylin and eosin (H&E) staining, tumors and indicated organs were fixed overnight in 4% paraformaldehyde (PFA). After tissue processing using standard procedures, samples were embedded in paraffin and cut into 3-1 μm sections followed by H&E staining. For immunofluorescence studies, samples were fixed in 1% PFA, dehydrated in 20% sucrose solution overnight, and embedded in tissue freezing medium (Leica). Frozen blocks were cut into 50-1 μm sections. Samples were blocked with 5% goat (or donkey) serum in PBST (0.03% Trition X-100 in PBS) and then incubated for 3 hr at room temperature (RT) with the following primary antibodies: anti-GFP (rabbit polyclonal, Millipore), anti-CD31 (hamster, clone 2H8, Millipore), anti-RhoJ (mouse, clone 1E4, Novus), anti-RhoJ (mouse, clone 1D7, OriGene), FITC-conjugated anti-c′-SMA (mouse, clone 1A4, Sigma-Aldrich), anti-VE-cadherin (rat, clone 11D4.1, BD Phamingen), anti-VE-cadherin (rabbit, clone D87F2, Cell Signaling), anti-PDGFRβ (rat, eBioscience), anti-Ter119 (rat, clone TER-119, eBioscience), anti-caspase-3 (rabbit polyclonal, R&D systems), anti-LYVE-1 (rabbit polyclonal, Angiobio), anti-pan-cytokeratin (mouse, clone AE1/AE3, Abcam), anti-melanin-A (rabbit polyclonal, Abcam), anti-collagen type IV (rabbit polyclonal, Cosmo Bio), or anti-cisplatin modified DNA (rat monoclonal, Abcam). After several washes, the samples were incubated for 2 hr at RT with the following secondary antibodies: FITC-, Cy3-, or Cy5-conjugated anti-hamster IgG (Jackson ImmunoResearch), FITC- or Cy3-conjugated anti-rabbit IgG (Jackson ImmunoResearch), Cy3-conjugated anti-rat IgG (Jackson ImmunoResearch), or Cy3-conjugated anti-mouse IgG (Jackson ImmunoResearch). Goat Fab fragment anti-mouse IgG (Jackson ImmunoResearch) was used to block endogenous mouse IgG to use mouse antibody on mouse tissues. F-actin was stained with acti-stain 555 phalloidin (Cytoskeleton). Nuclei were stained with 4′,6-diamidino-2-phenylindole (DAPI, Invitrogen). Then the samples were mounted with fluorescent mounting medium (DAKO) and immunofluorescent images were acquired using a Zeiss LSM510 confocal microscope (Carl Zeiss). To detect the hypoxic areas in the tumors, Hypoxyprobe-1™ (60 mg/kg, solid pimonidazole hydrochloride, Natural Pharmacia International) was IV injected 90 min before perfusion-fixation. The tumors were then harvested, sectioned, and stained with FITC-conjugated anti-Hypoxyprobe antibody.

### Example 1.9

### In Vivo Vascular Leakage and Perfusion Assay

At indicated days, tumor vessel leakage was analyzed after IV injection of 100 μl of FITC or rhodamine-conjugated dextran (25 mg/ml, 70 kDa, Sigma-Aldrich) 30 min before sacrifice. For vascular perfusion studies, 100 μl of DyLight® 594-conjugated tomato lectin (1 mg/ml, Vector laboratory) was IV injected 10 min before sacrifice. Mice were anesthetized and perfused by intracardiac injection of 1% PFA to remove circulating dextran and lectin.

### Example 1.10

### Morphometric Analyses

Density measurement of blood vessels, metastasis, hemorrhagic area, leakage area, and perfusion area were performed with ImageJ software (http://rsb.info.nih.gov/ij). For blood vessel density, CD31⁺ area per random 0.42 mm² areas was measured in the peri- and intratumoral regions. To determine the amount of blood vessels that express RhoJ, GFP⁺&CD31⁺ area per CD31⁺ area in five random 0.42 mm² areas were calculated. In addition, to find the percentage of CD31⁺ endothelial cells within RhoJ-GFP expressing cells, the GFP⁺&CD31⁺ area per total GFP⁺ area was also calculated. The measurements of metastasized cytokeratin⁺ cells in the lymph nodes were made on the total mid-section area. The area of cytokeratin⁺ fluorescence was presented as % per total sectioned area of lymph node. The measurements of hemorrhagic, necrotic and viable areas of tumors were made on the total mid-section area. The extent of hemorrhage was measured as a % of Ter-119⁺ area per random 0.24 mm² areas. Vascular leakage was quantified as the dextran⁺ area % per random 0.42 mm². Vascular perfusion area was calculated as the percentage of lectin⁺ area divided by CD31⁺ area in random 0.42 mm² regions. As for the quantification of lung metastasis, only the tumor colonies >100 μm in diameter were enumerated. Coverage of α-SMA⁺ or PDGFRβ⁺ mural cells and collagen-IV⁺ BM was calculated as the percentage of corresponding fluorescent positive length along the CD31⁺ vessels in random 0.42 mm² intratumoral regions. The numbers of vascular sprouts (>10 μm in length from the site of protrusion to the tip) were measured in the random 1 mm² peri- and intratumoral areas. The extent of cisplatin retention was measured as a % of cisplatin-modified DNA⁺ area per random 0.24 mm² areas. The photographs of remaining wound area were quantified using ImageJ software. The granulation area was measured as the cross-sectional granulation area (mm²) per total wound area. The calculation was performed using the middlemost section of the wound stained with H&E. All measurements were performed at least five different fields per mice.

### Example 1.11

### Flow Cytometry Analysis and Sorting

Tumor samples and lungs were harvested, chopped into small pieces, and digested into single cell suspension by incubating in digestion buffer (0.1% collagenase type 4 (Worthington) and 3 U/ml DNase I (Worthington)) for 1 hr at 37° C. The digested cells were filtered with a 40 μm nylon mesh to remove cell clumps. Cells were incubated for 10 min with the following antibodies in FACS buffer (5% bovine serum in PBS): PE-conjugated anti-mouse CD31 (rat, clone MEC13.3, eBioscience) and APC-conjugated anti-mouse CD45 (rat, clone 30-F11, eBioscience) antibodies. After several washes, the cells were analyzed and sorted by FACS Aria II (Beckton Dickinson). The purity of the sorted cells was at least 95%. Dead cells were excluded using 7-aminoactinomycin D (7-AAD, Invitrogen).

### Example 1.12

### Transcript Analysis by Quantitative Real-Time PCR

Total RNA was extracted from cultured cells and purified cells from tumors using RNeasy plus micro kit (Qiagen) according to the manufacturer's instructions. The extracted RNA was reverse transcribed into cDNA using SuperScript® II Reverse Transcriptase (Invitrogen). Quantitative real-time PCR was performed with indicated primer pairs listed below by using fast SYBR® green master mix (Roche) and CFX 96 Real-Time PCR Detection System (Bio-Rad™). The real-time PCR data were analyzed with CFX Manager Software (Bio-Rad™).

### Example 1.13

### siRNA Knockdown of RhoJ

HUVECs were purchased from Lonza, cultured in endothelial growth medium (EGM-2, Lonza) and incubated in a humidified atmosphere with 5% CO₂ at 37° C. The cells used were between passages 3 to 8. Transfections of siRNA duplexes into HUVECs were performed using Lipofectamine® RNAiMAX (Invitrogen) at a final concentration of 40 nM according to the manufacturer's protocol.

### Example 1.14

### Preparation and Application of APT_{EDB}-LS Complex for in-vivo siRNA Delivery

### Example 1.15

### Aptide specific for the EDB (APT_{EDB}) with the sequence CSSPIQGSWTWENGKWT (SEQ ID NO:6)

WGIIRLEQ (SEQ ID NO:7) was screened by phage-display technology and was synthesized (Anygen Corp) (Kim et al., 2012). The conjugation of APT_{EDB} and Mal-PEG2000-DSPE was carried out for 12 hr in RT, in which the molar ratio of was 1:2. The conjugation efficiency was then confirmed using a MALDI-TOF. For the preparation of anionic liposomes, POPC:Chol:POPG (molar ratio, 4:3:3; +/N/− charge ratio, 6:1:6) was added to make a lipid film as described previously (Saw et al., 2010). For APT_{EDB} targeting liposomes, 2.5 wt % of APT_{EDB}-PEG₂₀₀₀-DSPE were added to the original liposome. For Cy5.5 labeled liposomes, thiol-modified Cy5.5 (Lumiprobe) was conjugated with Mal-PEG₂₀₀₀-DSPE with the same protocol described above. Finally, to encapsulate RhoJ siRNA into the APT_{EDB}-liposome core, RhoJ siRNA was first complexed with 9R at 1:4 N/P ratio in HBG 5% buffer as described previously (Saw et al., 2010). 30 minutes after complexation, the complex was then added into the lipid film. To verify the successful delivery of APT_{EDB}-liposome into tumors, near-infrared images were taken at the indicated time points using an IVIS imaging machine (Xenogen). The effective knockdown of RhoJ in tumor tissue was confirmed using Immunoblotting.

### Example 1.16

### In Vitro EC Migration Assay

For EC migration assay, HUVECs were plated on a cell culture plate at 20% confluency. After 12 hr, migration of HUVECs was recorded as time-lapse movies. A Chamide magnetic chamber (Live Cell Instrument, Seoul, Korea) was kept at 37° C. and 5% CO₂ during experiment. An Axiovert 200M microscope (Carl Zeiss) equipped with an AxioCam MRm (Carl Zeiss) was used. Phase contrast images were acquired every 3 min for 6 hr. Migration patterns and speeds of HUVECs were analyzed by ImageJ software.

### Example 1.17

### Angiogenesis Assay Using A 3D Microfluidics System

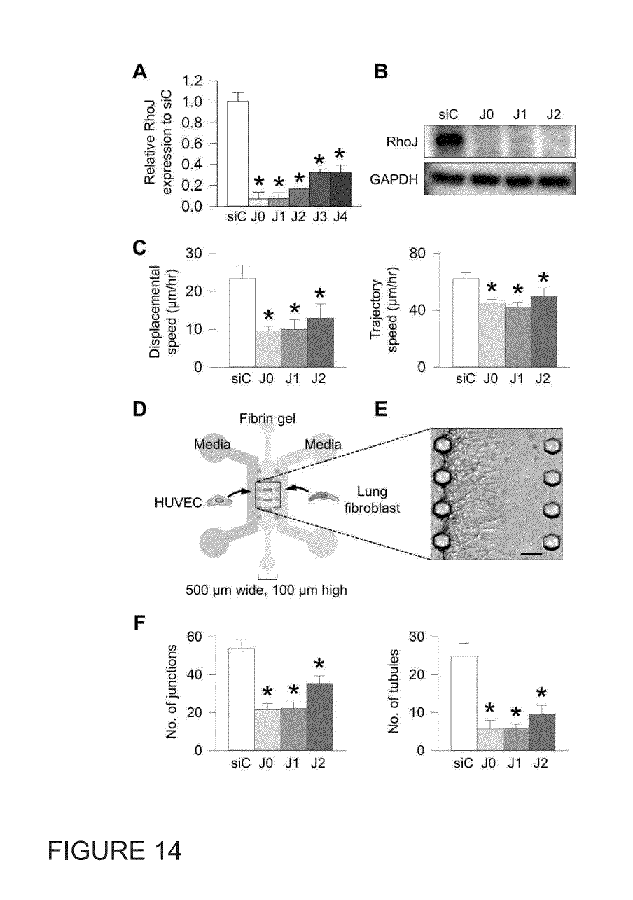

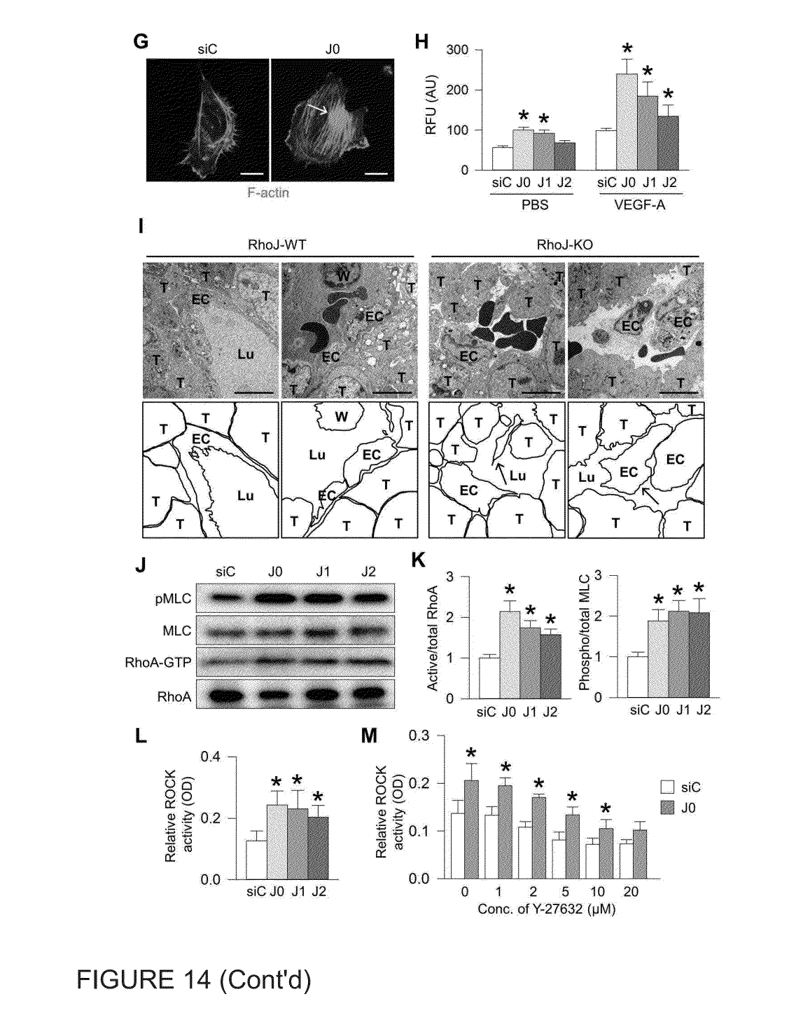

To evaluate the directional migration and angiogenic sprouting of ECs, we applied a 3D microfluidics system which we modified from the previously employed device (FIG. 14D) (Joo et al., 2012). The microfluidics system consists of 3 channels. Central channel was filled with fibrin gel, which is 500 μm wide and 100 μm high. Human lung fibroblasts (Lonza) were seeded with fibrin gel in the right channel in order to generate a concentration gradient with the growth factors that are secreted from fibroblasts. HUVECs were plated in the left channel and were allowed to migrate and sprout through the fibrin gel scaffold along the growth factor gradient toward the fibroblasts for 3 days (FIG. 14E).

### Example 1.18

### In Vitro Tube Formation Assay

For matrigel tube formation assay, growth factor reduced Matrigel (BD bioscience) was thawed overnight at 4° C. The Matrigel was allowed to solidify on a 4-well culture dishes at 37° C. for 30 min. Cells were harvested and seeded at a density of 2×10⁴ cells/well in growth media. Cells were then incubated at 37° C. for a further 12 hr. Tube formation was observed by taking pictures using a Leica DM IL microscope. The matrigel assays were quantified by counting the number of nodes and tubules from five different fields for each condition.

### Example 1.19

### In Vitro Vascular Permeability Assay

For in vitro vascular permeability assay, HUVECs were cultured on collagen-coated 1.0 μm-size pore insert (Millipore). After starvation for 12 hr, the cells were treated with or without VEGF (50 ng/ml) or CA4P (20 nM) for 2 hr and was then incubated with 70 kDa FITC-Dextran for 20 min. Each solution in plate wells were read with a Victor X2 multilabel plate reader.

### Example 1.20

### RhoA activity assay

RhoA activities were determined using a RhoA activation assay kit (BK036, Cytoskeleton). HUVECs were cultured at 40% confluence, starved overnight, and treated with VEGF-A (50 ng/ml) for 10 min. Cells were lysed and the cell lysates were incubated with the rhotekin beads (50 μg/sample) for 1 h at 4° C., washed two times, and eluted with Laemmli sample buffer. Bound RhoA, which is an active form of RhoA, was analyzed by SDS-PAGE separation followed by immunoblotting with an anti-RhoA antibody (ARH03, Cyto skeleton). The amount of total RhoA was also analyzed by immunoblotting using the same antibody in order to normalize the relative activity of RhoA.

### Example 1.21

### ROCK Activity Assay

ROCK activities were determined using a ROCK activity assay kit (CSA001, Millipore). HUVECs were lysed and cell lysates were incubated for 1 hr at room temperature in 96-well plates pre-coated with recombinant MYPT1, which contain Thr696 residue that can be phosphorylated by active ROCK. The plates were washed and incubated with anti-phospho-MYPT1 (Thr696) antibody followed by incubation with HRP-conjugated secondary antibody and HRP substrate reagent. The relative amount of active ROCK was measured by a microplate reader at 450 nm (Bio-Rad).

### Example 1.22

### Western Blotting

At the indicated time, tumor tissues or cultured HUVECs were homogenized in ice-cold lysis buffer containing a protease inhibitor cocktail (Roche). Each protein was separated with SDS-PAGE and transferred to PVDF membranes. After blocking with 5% skim milk, the membranes were incubated with the following primary antibodies in blocking buffer overnight at 4° C.: RhoJ (mouse, clone 1D4, Novus), ROCK1 (mouse, clone G-6, Santacruz), pMLC (rabbit polyclonal, Cell signaling), MLC (rabbit polyclonal, Cell signaling), RhoA (mouse monoclonal, Cytoskeleton), GAPDH (rabbit polyclonal, Santacruz), and β-actin (rabbit polyclonal, Sigma). Membranes were then incubated with HRP-conjugated secondary antibodies for 2 hr at RT. Chemiluminescent signals were developed with HRP substrate (Millipore) and detected with a LAS-1000 mini (Fuji film).

### Example 1.23

### Immunofluorescent Staining of ECs

HUVECs were cultured on glass coverslips coated with 0.1% gelatin for overnight. Cells were fixed with 1% PFA and permeablized with ice cold 0.3% PBST for 5 min and blocked in 5% goat serum in 0.1% PBST for 1 hr at room temperature. Samples were incubated with VE-cadherin antibody (rabbit, clone D87F2, Cell Signaling) for 3 hr at room temperature. After several washes, Cells were incubated for 2 hr with the FITC-conjugated anti-mouse IgG (Jackson ImmunoResearch). Nuclei were stained with DAPI (Invitrogen) and F-actin was stained with Acti-stain™ 488 Phalloidin (Cytoskeleton). Then the cells were mounted and analyzed using a LSM 510 confocal microscope (Zeiss).

### Example 1.24

### Electron Microscopy

Tissues (LLC tumor, heart, lung, liver, and kidney) and HUVECs were fixed with 2.5% glutaraldehyde in PBS overnight and washed with cacodylate buffer (0.1 M) containing 0.1% CaCl₂. Samples were post-fixed for 2 hr with 1% OsO₄ in cacodylate buffer (pH 7.2) and washed with cold distilled water. Dehydration was performed with ethanol series and propylene oxide. Samples were embedded in Embed-812, resin polymerized, sectioned, and mounted on a formvar-coated slot grid. After staining with 4% uranyl acetate and lead citrate, sample images were acquired with a Tecnai G2 Spirit Twin transmission electron microscope (FEI).

### Example 1.25

### Statistical Analyses

Values are presented as mean±standard deviation. Statistical differences between means were determined by unpaired Student t-test or analysis of variance with one-way followed by the Student-Newman-Keuls test. Chi-square test was used to analyze discrete variables. The survival curve was evaluated using the Kaplan-Meier method, and statistical differences were analyzed using the log-rank test. Bivariate correlation was evaluated by the two-tailed Pearson test. Statistical significance was set at p<0.05.

List of Primer Sets for Quantitative Real-Time PCR

| Name | Sequence (5′- 3′) | |
|---|---|---|
| mouse | Forward | 5′-GGGAAGACCTGCTTGCTGATGAG-3′ |
| RhoJ | | (SEQ ID NO: 8) |
| | Reverse | 5′-GCAGTGTCGTATAGTCCAAGCAAGTG-3′ |
| | | (SEQ ID NO: 9) |
| mouse | Forward | 5′-CACGGTGAAGGACAGTGGAGAGTATG-3′ |
| PDGFRα | | (SEQ ID NO: 10) |
| | Reverse | 5′-CTTCGTGCAAGTTGACAGCTTCCAG-3′ |
| | | (SEQ ID NO: 11) |
| mouse | Forward | 5′-GCTGCTGGAGACACTGGGAGATG-3′ |
| PDGFRβ | | (SEQ ID NO: 12) |
| | Reverse | 5′-GGAGTCACCCAAGGTACGGTTGTC-3′ |
| | | (SEQ ID NO: 13) |
| mouse | Forward | 5′-CATCGTGGGACGTCCCAGACATCA-3′ |
| α-SMA | | (SEQ ID NO: 14) |
| | Reverse | 5′-ACAGCCTGAATAGCCACATACATGGC-3′ |
| | | (SEQ ID NO: 15) |
| mouse | Forward | 5′-CACAAATACTCAGGCAAAGAGGGTGAC-3′ |
| FSP-1 | | (SEQ ID NO: 16) |
| | Reverse | 5′-CTGTTGCTGTCCAAGTTGCTCATCAC-3′ |
| | | (SEQ ID NO: 17) |
| mouse | Forward | 5′-GTCGTGGAGTCTACTGGTGTCTTCAC-3′ |
| GAPDH; | | (SEQ ID NO: 18) |
| | Reverse | 5′-GTTGTCATATTTCTCGTGGTTCACACCC-3′ |
| | | (SEQ ID NO: 19) |

List of siRNA Sequences

| Name | Sequence (5′- 3′) |
|---|---|
| Control | UAGCGACUAAACACAUCAA (SEQ ID NO: 20) |
| (SiC) | |
| Human RhoJ | GCUGUAUAUGAAAGAGAAA (SEQ ID NO: 21) |
| (J0) | |
| Human RhoJ | CCACTGTGTTTGACCACTA (SEQ ID NO: 22) |
| (J1) | |
| Human RhoJ | AGAAACCTCTCACTTACGA (SEQ ID NO: 23) |
| (J2) | |
| Human RhoJ | UCAUAGGGACCCAGAUUGA (SEQ ID NO: 24) |
| (J3) | |
| Human RhoJ | UCAGAAAGGUCUCAAAGCG (SEQ ID NO: 25) |
| (J4) | |
| Mouse RhoJ | GCGCAGTGCTACTTGGAAT (SEQ ID NO: 26) |

### Example 2

### Results

### Example 2.1

### High Expression of RhoJ in Tumor ECs during Tumor Progression

To unveil the role of RhoJ in tumor progression, we generated RhOj^{GFP/GFP} (RhoJ-KO) mice, in which Rhoj is knocked out by replacing its exon 1 with GFP; with this construct, GFP is expressed instead of Rhoj under the transcriptional control of the Rhoj promoter (FIGS. 9A, 9B, and 9C). To monitor RhoJ expression in tumor tissues, Rhoj^{GFP/+} mice were implanted with Lewis lung carcinoma (LLC) and B16F10 melanoma cells. To observe RhoJ expression in a spontaneous breast tumor model, we generated MMTV-PyMT/Rhoj^{GFP/+} mice (P/Rhoj^{GFP/+}) by mating Rhoj^{GFP/+} mice with MMTV-PyMT mice. LLC tumor and B16F10 melanoma displayed high RhoJ expression in tumor vessels 7 days after implantation, and spontaneous breast tumors of P/Rhoj^{GFP/+} also showed strong RhoJ expression in tumor vessels 12 weeks after birth (FIG. 1A). In contrast to robust expression in tumor vessels, RhoJ expression was not observed in the lymphatic vessels of tumors and lymph nodes (LNs). High-magnification analyses of the LLC tumor revealed that RhoJ expression was mainly confined to tumor ECs, while some non-ECs such as perivascular mural cells and tumor stromal cells also occasionally expressed RhoJ (FIG. 1B). qRT-PCR analysis of purified cells from LLC tumors showed that they consistently exhibited a predominant expression of RhoJ in CD31⁺CD45⁻ tumor ECs with a weak expression in CD31⁻CD45⁻ cells but no expression in CD31⁻ CD45⁺ hematopoietic cells (FIG. 1C). To identify RhoJ-expressing stromal cells other than ECs, GFP and GFP⁻ cells were purified from the CD31⁻CD45⁻ cells of P/Rhoj^{GFP/+} tumors using FACS (FIG. 9D). We discovered that these RhoJ-expressing non-ECs highly expressed PDGFRα, PDGFRβ, α-SMA, and FSP-1, indicating that these cells could be pericytes and cancer-associated fibroblasts (FIG. 9E). In tumor vasculature, RhoJ expression follows a distinct spatiotemporal regulation. It is most robustly expressed during early tumorigenesis, in contrast to being attenuated in later stages of tumor growth (FIGS. 1C, 1D, and 1E). Moreover, RhoJ is intensively expressed in the peritumoral high-angiogenic region compared to the intratumoral regions of various tumors (FIGS. 1F, 1G, and 9F). Intriguingly, RhoJ expression in normal tissues of adult mice is very infrequent and indistinct, only being occasionally present in heart blood vessels and stromal cells and in LN blood vessels (FIG. 9G). RhoJ-KO mice grew to adulthood normally without any growth retardation or vascular abnormality in major organs including heart, lung, kidney and liver (FIG. 9H). Also there were no differences in vascular morphology and integrity between RhoJ-KO mice and wild-type (WT) mice (FIGS. 91-0). These findings suggest that RhoJ is a potential candidate for a more selective vascular targeting therapy with attenuated systemic side effects compared to current AIA therapies.

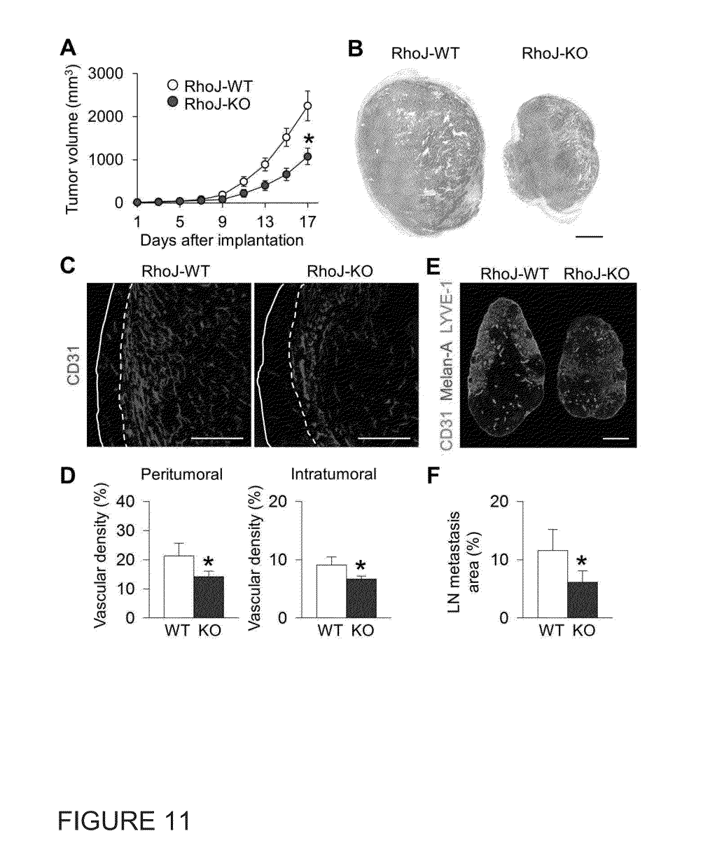

To examine the relevance of RhoJ in human tumor angiogenesis, we assessed RhoJ expression in human tissues and confirmed that RhoJ is highly expressed in the tumor vessels of colon adenocarcinomas (7 of 12 samples) but is undetectable in normal colon tissues (0 of 10 samples) (FIG. 1H). Furthermore, we analyzed the RhoJ expression using the 216 colon cancer patients dataset of The Cancer Genome Atlas (http://cancergenome.nih.gov) (Table 1) and found that the patients having tumors with high RhoJ expression had increased prevalence of lymphovascular invasion (FIG. 11) and had decreased overall survival after the diagnosis of colon cancer (FIG. 1J). Finally, the RhoJ expression positively correlated with the number of metastatic LNs (FIG. 1K), suggesting the possible positive correlation of RhoJ with human cancer progression.

### Example 2.2

### RhoJ Deletion Suppresses Tumor Growth, Neovessel Formation, and Metastasis in the LLC Tumor

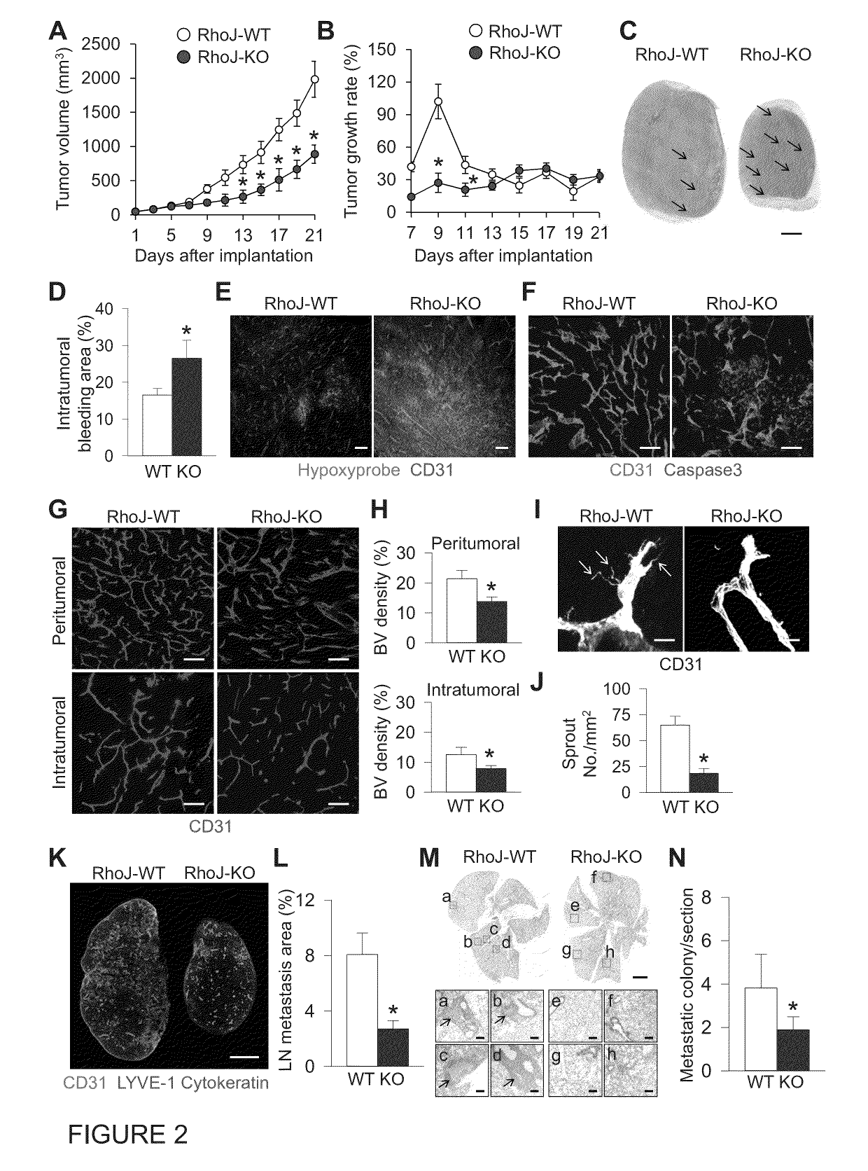

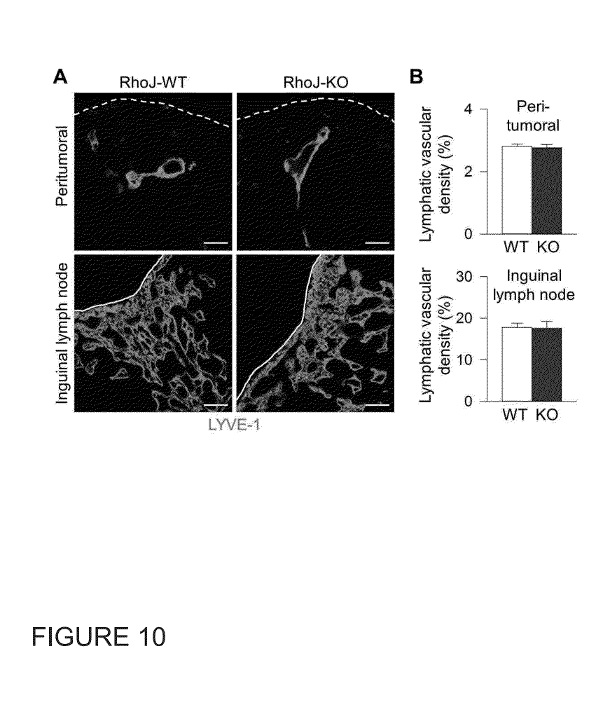

Taking the advantage that RhoJ-KO mice grew to adulthood normally, we used RhoJ-KO mice to address the role of RhoJ during tumor progression. We employed the LLC tumor model by subcutaneously (SC) injecting LLC cells into RhoJ-WT and KO mice. At 3 weeks after implantation, compared to WT mice, RhoJ-KO mice showed a 55% reduced tumor growth (FIG. 2A), which was most prominent during early growth (FIG. 2B). Tumors had an increased occurrence of hemorrhagic foci in RhoJ-KO mice (FIG. 2C), in which the intratumoral hemorrhagic area was 61% higher than WT (FIG. 2D). Moreover, hypoxia was more apparent with extensive apoptosis in the center of the tumor in RhoJ-KO mice (FIGS. 2E and 2F). To further evaluate the impact of RhoJ deletion on tumor angiogenesis, we investigated tumor vessels at 2 weeks after implantation. Compared to RhoJ-WT mice, tumor vascular densities in RhoJ-KO mice were 36% and 37% less in the peri- and intratumoral regions, respectively (FIGS. 2G and 2H), while there were no differences in the distribution and densities of lymphatic vessels at the peritumoral regions and sentinel LNs (FIGS. 10A and 10B). Most importantly, tumor vascular sprouting was 72% lower in RhoJ-KO mice (FIGS. 2I and 2J), suggesting that RhoJ is crucial in neovessel formation by promoting sprouting angiogenesis. Finally, to examine the role of RhoJ in tumor metastasis, we harvested inguinal LNs and whole lungs of the tumor-bearing mice 3 weeks after tumor implantation. The analyses showed 66% less metastasized LLC tumor cells in the LNs of RhoJ-KO mice (FIGS. 2K and 2L). Moreover, the number of metastatic tumor colonies (>100 μm in diameter in tumor sections) in the lungs was 51% less in RhoJ-KO mice (FIGS. 2M and 2N).

### Example 2.3

### RhoJ Deletion Disrupts Tumor Vascular Integrity and Function

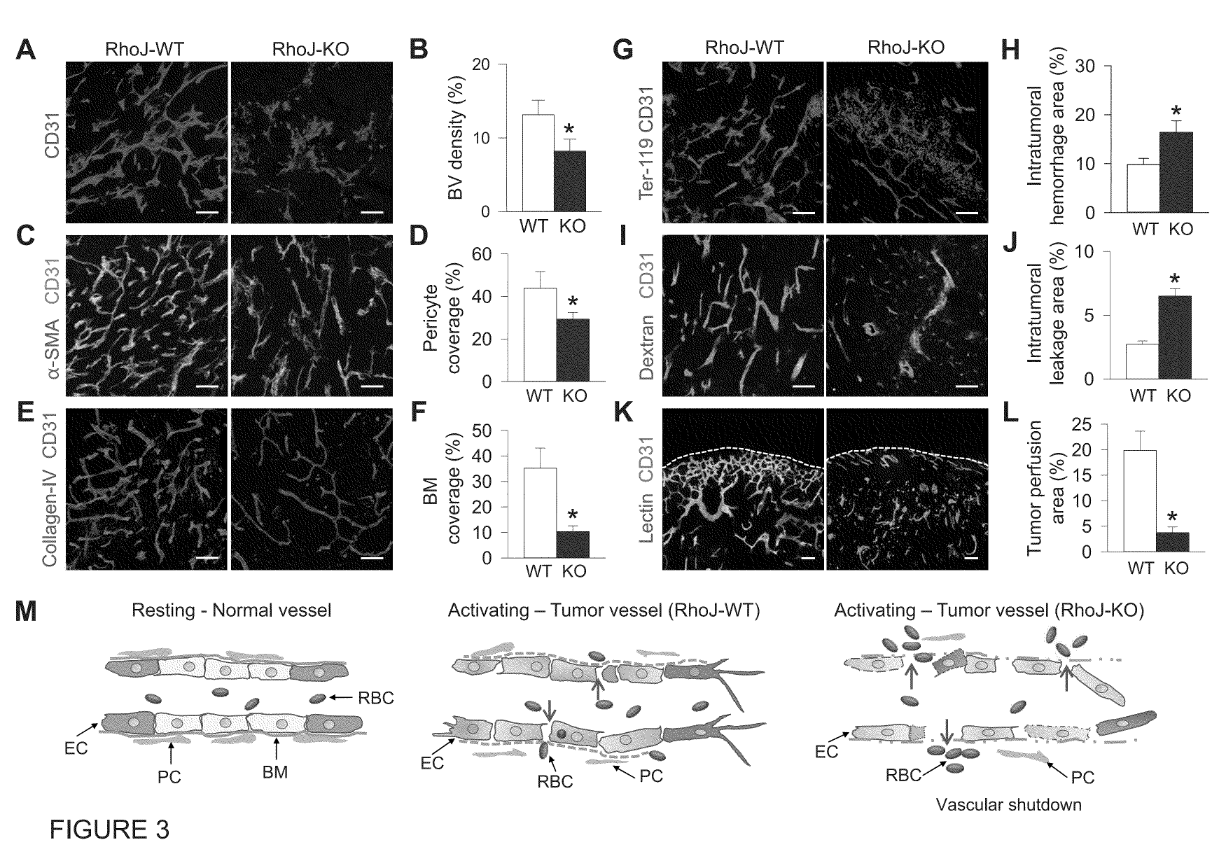

Tumor vasculature consists of malformed, disintegrated, leaky and highly branched vessels that continuously undergo vascular remodeling (McDonald and Baluk, 2002; Siemann, 2011; Trédan et al., 2007). Because RhoJ-KO mice displayed increased intratumoral hemorrhage compared to RhoJ-WT mice, we further investigated the role of RhoJ in vascular integrity and function. Interestingly, LLC tumor of RhoJ-KO mice had more disrupted tumor vessels (FIG. 3A) and reduced vascular density (FIG. 3B) in the center. Together with this observation, coverage of α-SMA⁺ mural cells alongside tumor vessels was 33% less (FIGS. 3C and 3D) and collagen type IV⁺ basement membrane (BM) coverage was reduced by 71% in tumor vessels of RhoJ-KO mice (FIGS. 3E and 3F), revealing that RhoJ-KO tumor vessels were more disintegrated with severe loss of pericytes and BM components. Consistent with these findings, RhoJ-KO tumor vessels displayed a 67% increased intratumoral hemorrhage (FIGS. 3G and 3H) and more than 2-fold increase in dextran leakage where dextran was intravenously (IV) injected (FIGS. 31 and 3J), indicating a significant increase in tumor vascular permeability. We next evaluated the functionality of tumor vessels by IV injecting lectin. Tumor vascular perfusion was profoundly impeded by 81% in tumors of RhoJ-KO mice (FIGS. 3K and 3L). Altogether, these findings suggest that RhoJ-deficient tumor vessels are more disintegrated and permeable than typical tumor vessels and that the increased extravasation and hemorrhage eventually lead to severely retarded blood flow into tumor tissues (FIG. 3M). This scenario is quite comparable with the “vascular shutdown” phenomenon caused by vascular-disrupting agents (VDAs) (Tozer et al., 2005).

To determine whether RhoJ deletion affects tumor progression broadly, we also evaluated the melanoma model by SC implantation of B16F10 cells into RhoJ-WT and KO mice. Consistent with the findings observed in LLC tumors, tumor growth was delayed by 52% in RhoJ-KO mice compared to RhoJ-WT mice (FIGS. 11A and 11B). In terms of tumor angiogenesis, vascular densities were reduced by 31% and 28% in the peri- and intratumoral regions of Rho-KO mice, respectively (FIGS. 11C and 11D). Moreover, lymphatic metastasis of tumor cells into inguinal LNs was suppressed by 47% in RhoJ-KO mice (FIGS. 11E and 11F).

### Example 2.4

### RhoJ Deletion Also Reduces Tumor Growth, Neovessel Formation, and Metastasis in Spontaneous Breast Cancer Model

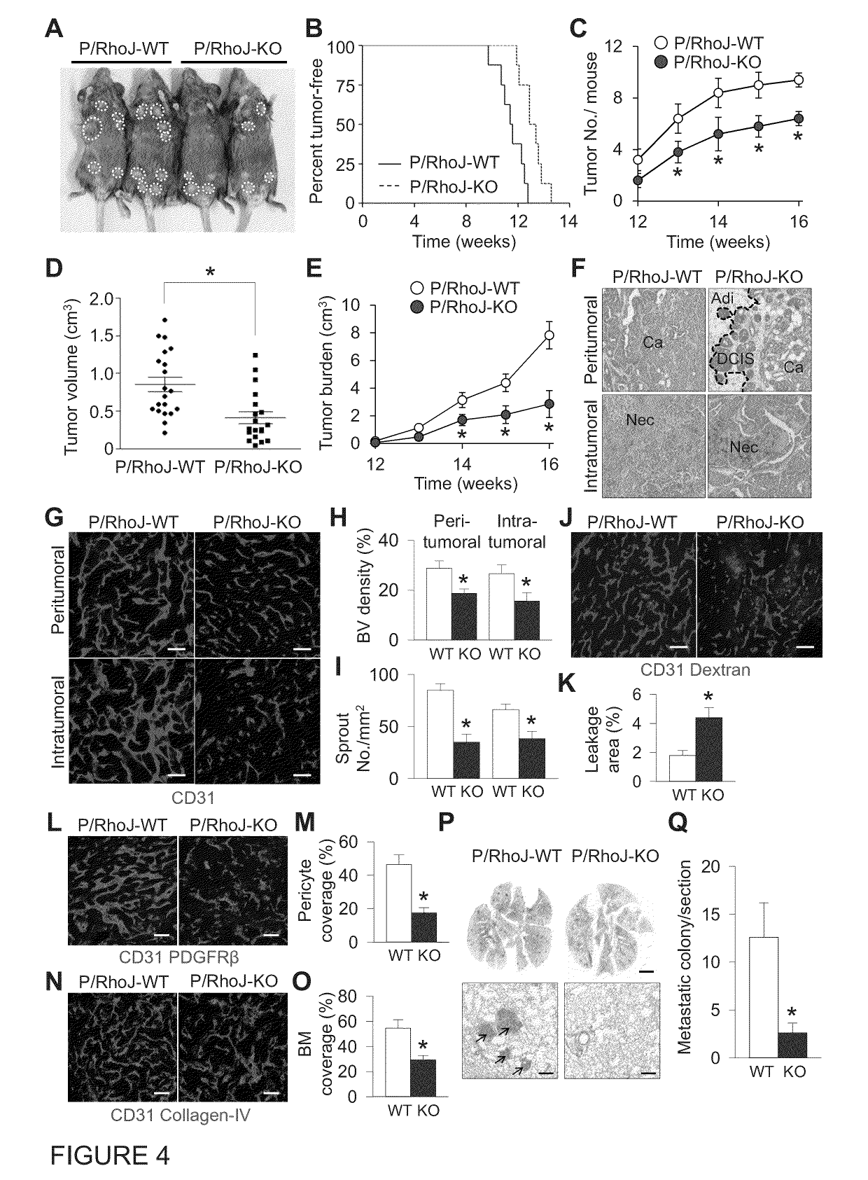

As for the spontaneous tumor model, MMTV-PyMT mice were mated with Rhoj^{GFP/+} mice to generate MMTV-PyMT;Rhoj^{+/+} mice (P/RhoJ-WT) and MMTV-PyMT;Rhoj^{GFP/GFP} mice (P/RhoJ-KO). At 14 weeks of age, P/RhoJ-KO showed reduced development of spontaneous mammary tumor nodules compared to P/RhoJ-WT (FIG. 4A). In the P/RhoJ-KO, compared to those in P/RhoJ-WT, median time to palpable tumor development was delayed by ˜2 weeks (FIG. 4B), and number of tumor nodules per mouse decreased 32-41% (FIG. 4C). Moreover, the average size and tumor burden of P/RhoJ-KO mice were 61% and 64% less, respectively, than those of P/RhoJ-WT mice (FIGS. 4D and 4E). Histological examination showed that there were more non-invasive carcinoma lesions with well-preserved tumor margins in the peritumoral regions of P/RhoJ-KO (FIG. 4F, see legend for a detailed explanation), which indicates that RhoJ deficiency delays tumor progression and invasion. Also, in P/RhoJ-KO, vascular densities were 35% and 41% less (FIGS. 4G and 4H) and tumor vascular sprouting was 59% and 42% less (FIG. 4I) in the peri- and intratumoral regions, respectively, compared to P/RhoJ-WT. Furthermore, morphology of tumor vasculatures in the intratumoral regions of P/RhoJ-KO seemed more disrupted (FIG. 4G). Consistent with their disintegrated morphology, extravasation of IV-injected dextran was 2.5-fold greater in the tumor vessels of P/RhoJ-KO (FIGS. 4J and 4K), indicating that P/RhoJ-KO tumor vessels are highly permeable compared to those of P/RhoJ-WT. In addition, PDGFRβ⁺ pericyte support was 62% less (FIGS. 4L and 4M) and the track of collagen type IV⁺ BM along tumor vessels was 46% less (FIGS. 4N and 4O) in P/RhoJ-KO. Finally, the number of metastatic tumor colonies (>100 μm in diameter in tumor sections) in the lung was 82% less in P/RhoJ-KO (FIGS. 4P and 4Q). Taken together, these results lead us to conclude that RhoJ plays a crucial role in the formation of tumor neovessels and maintenance of tumor vascular integrity, influencing the tumor progression.

### Example 2.5

### RhoJ Deficiency Delays Wound Healing through Attenuated

### Angiogenesis

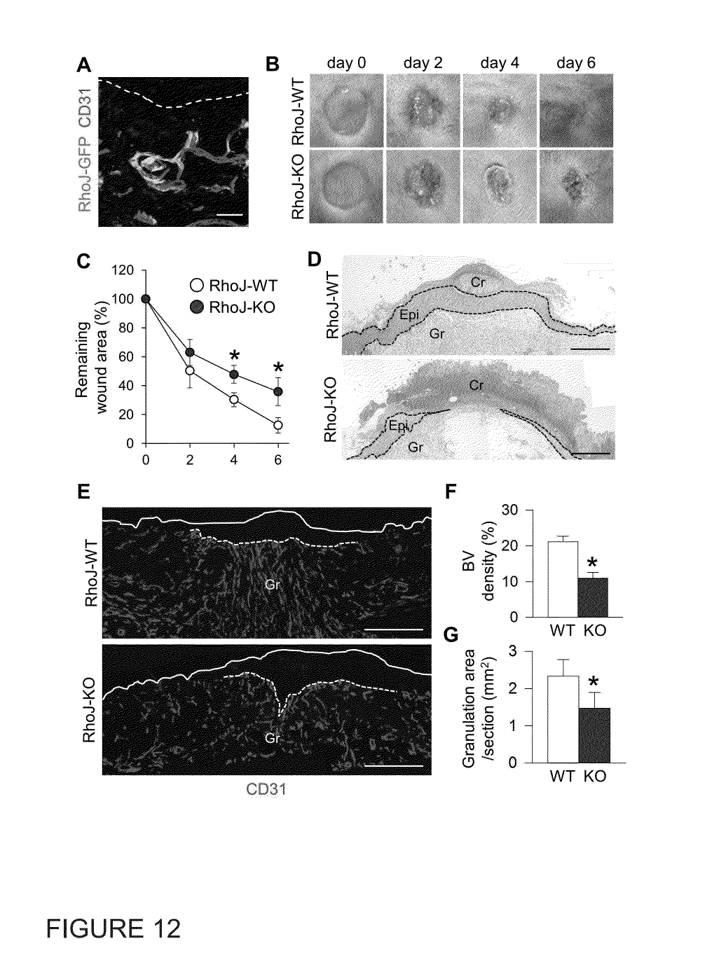

We also evaluated the role of RhoJ in wound healing using a punch-wound healing model. Like tumor vessels, the blood vessels in the granulation area of wounds displayed high RhoJ expression (FIG. 12A). Compared to RhoJ-WT mice, RhoJ-KO mice showed 23% delayed wound closure, 48% reduced vascular density, and 39% decreased granulation area in the wound regions (FIGS. 12B-G). Thus, RhoJ plays a positive angiogenic role in wound healing.

### Example 2.6

### Targeted RhoJ Deletion in Tumor ECs Suppresses Tumor Angiogenesis and Disrupts Tumor Vessel Integrity

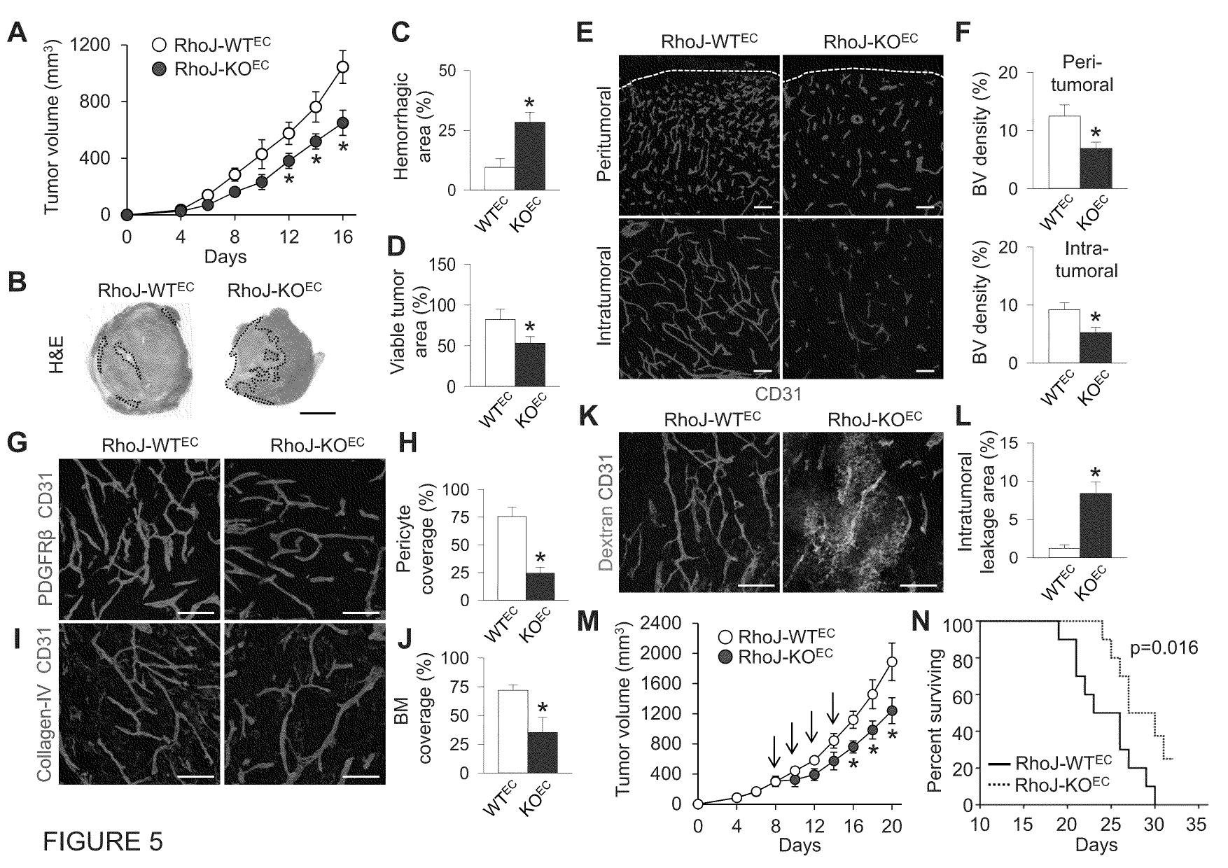

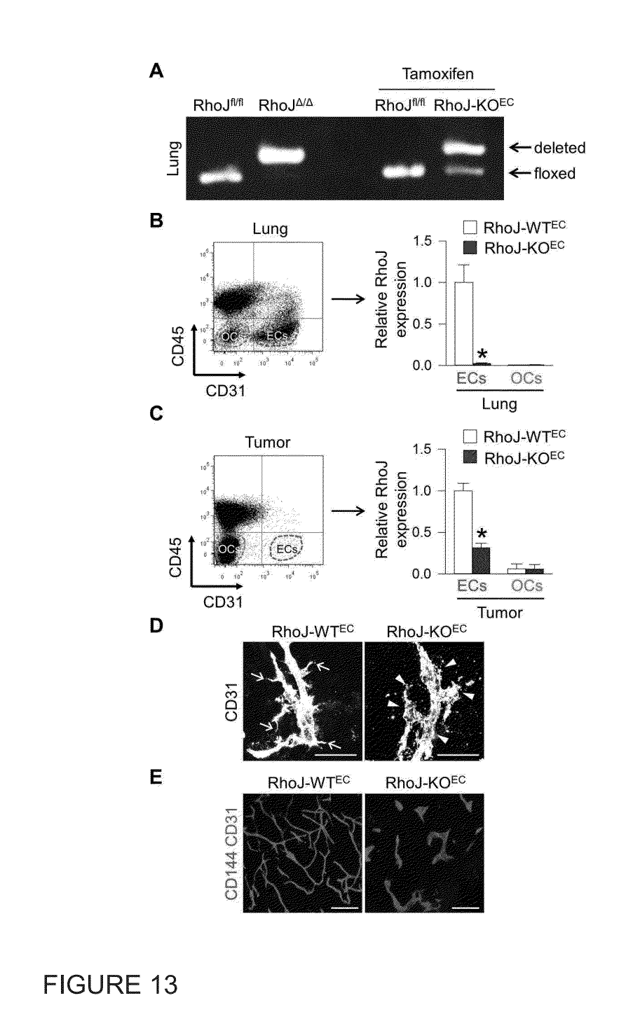

To ascertain the role of RhoJ in tumor ECs during tumor angiogenesis, we generated inducible EC-specific RhoJ loss-of-function mice (RhoJ-KO^{EC}) by mating Rhoj^{fl/fl} with Cdh5(PAC)-CreER^{T2} (Wang et al., 2010), in which the Rhoj allele was efficiently deleted in the ECs upon tamoxifen administration (FIGS. 13A-C). Rhoj^{fl/fl} mice (RhoJ-WT^{EC}) were used as control. Compared to those in RhoJ-WT^{EC}, LLC tumors showed 38% reduced growth in RhoJ-KO^{EC} (FIG. 5A) and remarkable intratumoral hemorrhagic necrosis 16 days after LLC tumor implantation (FIG. 5B), in which the hemorrhagic area was 3.0-fold larger and viable tumor areas were 35% less (FIGS. 5C and 5D). The impact of EC-specific Rhoj deletion on tumor growth was 31% less compared to global Rhoj deletion, which is attributed by the 70% deletion of RhoJ in tumor ECs (FIG. 13C). Vascular densities of RhoJ-KO^{EC} tumors were 45% and 43% reduced in the peri- and intratumoral areas, respectively (FIGS. 5E and 5F). Notably, the morphology of tumor vessels in the intratumoral core of RhoJ-KO^{EC} was more disrupted (FIG. 13D). Additionally, PDGFRβ⁺ pericyte coverage was reduced by 68% (FIGS. 5G and 5H), and collagen type IV⁺ BM coverage along tumor vessels was 51% diminished (FIGS. 5I and 5J) in RhoJ-KO^{EC}. Moreover, the leakage of IV-injected dextran was remarkably increased by 6.8-fold in the intratumoral core of RhoJ-KO^{EC} (FIGS. 5K and 5L). Finally, junctional CD144 expression seemed to be decreased in the intratumoral regions of RhoJ-KO^{EC} (FIG. 13E). We also evaluated the effect of endothelial RhoJ deletion on established macroscopic tumors (>300 mm³). The results showed that the tumor growth was decreased by 34% (FIG. 5M) and the overall survival of mice increased by ˜25% (FIG. 5N), denoting that RhoJ is a feasible target for further anti-cancer drug development even in established tumors. In conclusion, these findings indicate that RhoJ in tumor ECs is critical in regulation of tumor angiogenesis and maintenance of tumor vascular integrity.

### Example 2.7

### RhoJ Regulates EC Motility, Tube Formation, and Junctional Integrity through Suppression of the RhoA-ROCK Signaling Pathway

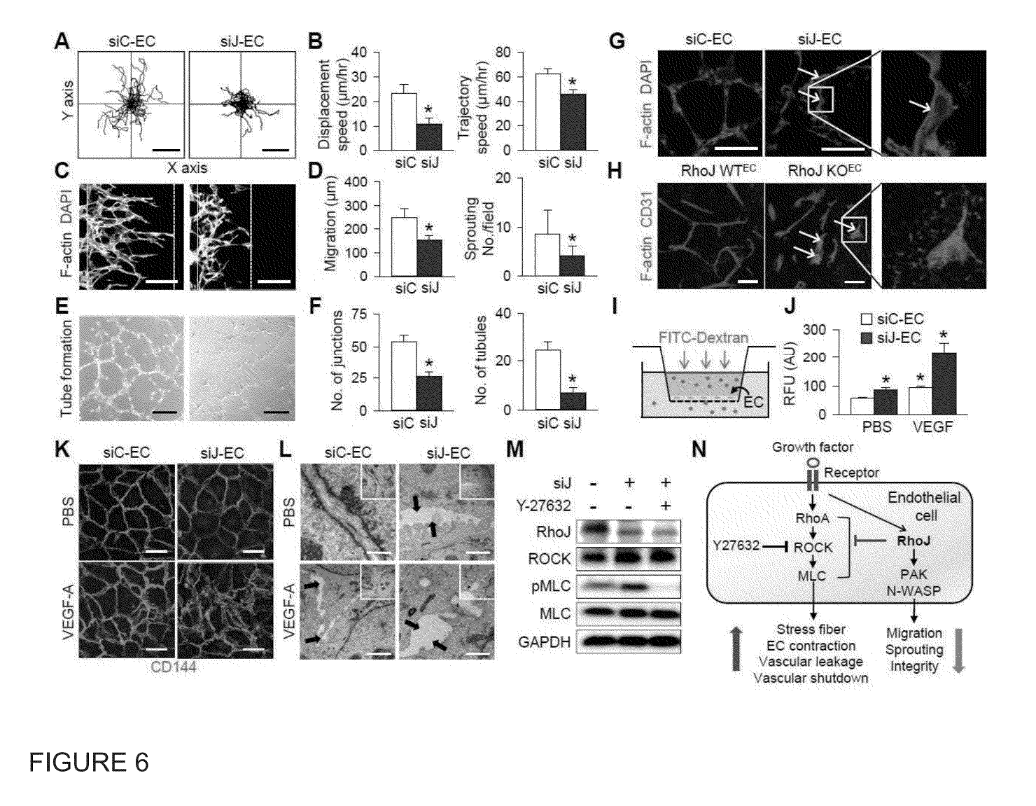

To determine the role of RhoJ in in vitro angiogenesis and vascular leakage, HUVECs transfected with either RhoJ siRNA (siJ-ECs) or control siRNA (siC-ECs) were used. To exclude the off-target effects, 5 independent RhoJ siRNA were designed, and 3 RhoJ siRNAs with the best performance (named J0, J1, and J2) was chosen for further experiments (FIGS. 14A and 14B) and their results were averaged (siJ-EC) (see FIG. 14 for their individual results). In comparison to those of siC-ECs, a time-lapse tracking analysis revealed that siJ-ECs displayed more restricted motility with a 54% reduction in displacement speed and 27% reduction in trajectory speed (FIGS. 6A, 6B, and 14C). Moreover, our established microfluidics assay (Joo et al., 2012) (FIGS. 14D and 14E) showed that siJ-ECs had 38% and 50% less migration and angiogenic sprouting, respectively (FIGS. 6C and 6D), indicating that RhoJ is an important regulator of EC migration and sprouting. Furthermore, siJ-ECs on the Matrigel formed poorly connected networks with decreased numbers of EC junctions and tubules (FIGS. 6E, 6F, and 14F). In addition, detailed analysis of EC and EC tubules showed that siJ-ECs have increased actin stress fiber formation (FIGS. 6G and 14G). In agreement with this result, increased actin stress fiber formation was also observable in the intratumoral vasculatures of RhoJ-KO^{EC} (FIG. 6H), confirming the negative correlation between RhoJ and EC stress fiber formation (Kaur et al., 2011).

We next questioned whether RhoJ has any role in maintaining EC integrity because various Rho GTPases are also involved in endothelial integrity (Beckers et al., 2010; Bryan and d'Amore, 2007). To answer this question, an in vitro vascular permeability assay was applied to examine the changes in EC paracellular integrity (FIG. 6I). Compared to siC-ECs, the vascular permeability across the EC monolayer was increased by 55% and 134% with or without VEGF-A, respectively in siJ-ECs (FIGS. 6J and 14H). Consistent with this finding, junctions between ECs were also more loose and disrupted in siJ-ECs, especially in the presence of VEGF-A (FIGS. 6K and 6L). In parallel, tumor blood vessels of RhoJ-KO mice were also seriously disrupted (FIG. 14I). These suggest that RhoJ works to maintain the integrity of the EC monolayer and negatively regulates VEGF-A-induced vascular leakage. Finally, we questioned whether RhoJ is associated with RhoA-ROCK-myosin signaling, since this signaling is an important regulator of stress fiber formation and EC contraction (Sun et al., 2006) and endothelial RhoJ also seemed to be related to the regulation of stress fibers. Indeed, siJ-ECs had increased RhoA activity, ROCK activity, and myosin light chain (MLC) phosphorylation (FIGS. 14J-L), but these almost completely diminished with the ROCK inhibitor, Y-27632 (FIGS. 6M and 14M), suggesting that RhoJ is a negative regulator of the RhoA-ROCK signaling pathway in ECs. Collectively, these findings indicate that RhoJ plays an important role in EC migration, tube formation, and maintenance of vascular integrity through the suppression of the RhoA-ROCK signaling pathway in ECs (FIG. 6N).

### Example 2.8

### Cisplatin Profoundly Retards Tumor Growth in RhoJ-Deleted Mice

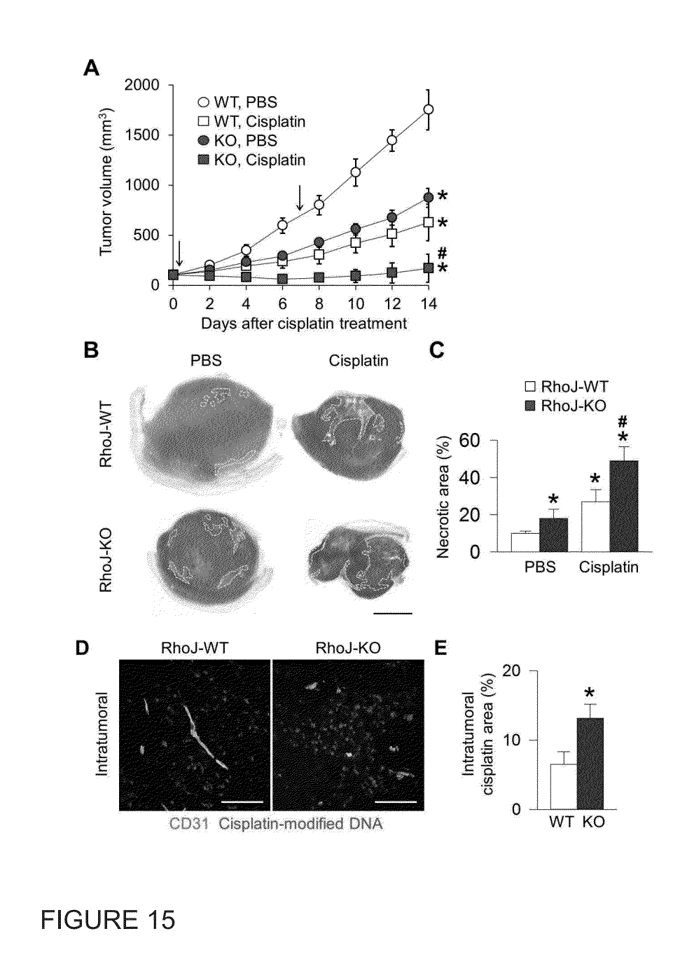

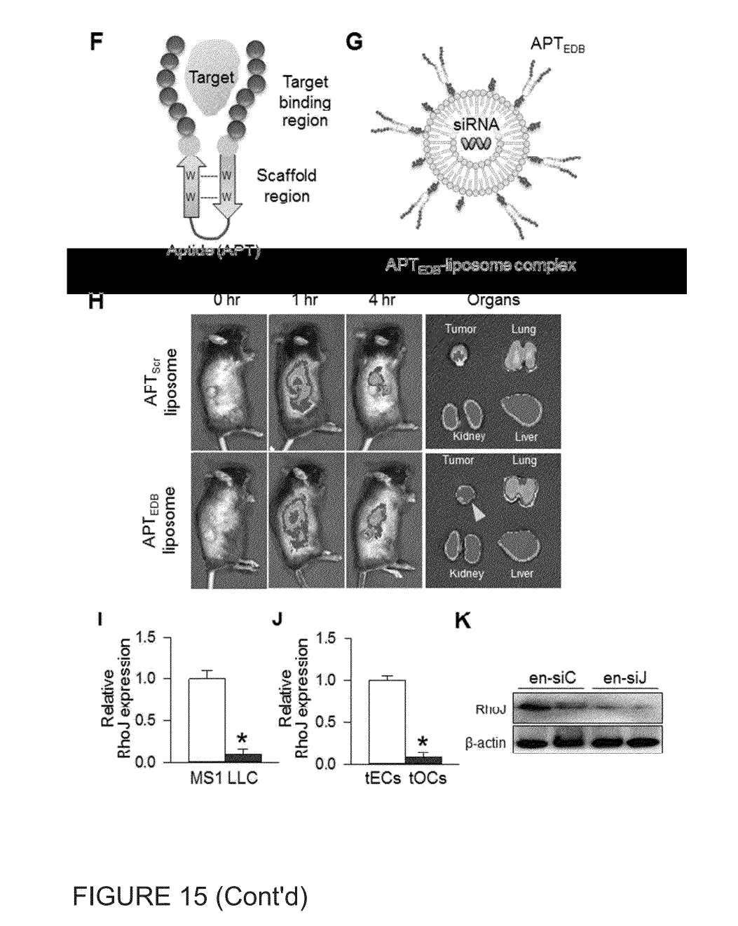

To confirm the effect of RhoJ deletion in concert with conventional chemotherapeutic drugs, cisplatin (10 mg/kg) was intraperitoneally (IP) injected into RhoJ-KO mice once every week starting when tumor volume exceeded 100 mm³. Cisplatin significantly delayed LLC tumor growth by 90% in RhoJ-KO mice compared to a 64% decrease in RhoJ-WT mice (FIG. 15A). Histological analyses after cisplatin treatment revealed 80% increased intratumoral necrosis in RhoJ-KO mice treated with cisplatin compared to RhoJ-WT mice treated with cisplatin (FIGS. 15B and 15C). In fact, intratumoral accumulation of cisplatin increased by ˜2.0-fold in RhoJ-KO mice compared to RhoJ-WT mice (FIGS. 15D and 15E). This may be due to increased extravasation and retention of cisplatin from the disintegrated tumor vessels at the intratumoral region of RhoJ-KO mice. These findings indicate that RhoJ blockade in combination with conventional chemotherapy could yield an enhanced anti-tumor effect.

### Example 2.9

### Dual Blockade of RhoJ and VEGF-A Signaling Displays More Potent Anti-Tumor Effect

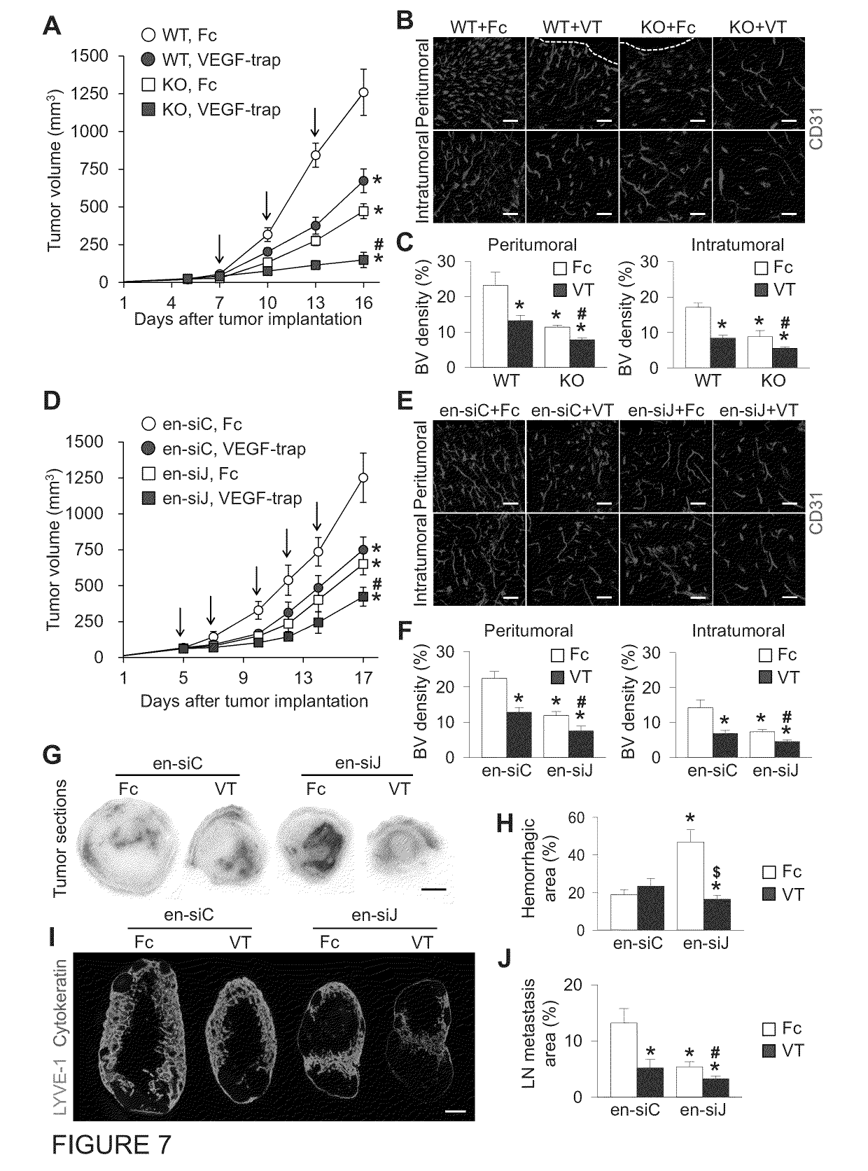

Many Rho GTPases are activated by VEGF-A and share their common downstream effector molecules (Beckers et al., 2010; Schiller, 2006). Therefore, there is a possibility that VEGF-A-driven activation of other Rho GTPases may partially compensate for the effects of RhoJ ablation, limiting the anti-tumor effects of the RhoJ blockade. To resolve this potential problem and maximize the anti-tumor effect, we investigated the effect of VEGF-A blockade in the tumor progression of RhoJ-WT and KO mice. Administration of VEGF-trap (25 mg/kg) delayed LLC tumor growth by 88% in RhoJ-KO mice compared to a 47% decrease in RhoJ-WT mice (FIG. 7A). Moreover, VEGF-trap reduced tumor vascular densities by 66% and 68% in peri- and intratumoral areas of RhoJ-KO mice, respectively, which was more potent than the 43% and 49% decrease in RhoJ-WT mice (FIGS. 7B and 7C). From these results, we could confirm the potential of RhoJ blockade as an adjuvant option to enhance AIA therapies, such as VEGF-trap.

Next, to establish a method for therapeutic blockade of RhoJ, a tumor-targeted siRNA delivery system (Kim et al., 2012) was employed. The aptide was designed and used according to a previous protocol (FIG. 15F) (Kim et al., 2012). We chose fibronectin extradomain B (EDB) as the target for the aptide because EDB is highly expressed in tumor tissues (Kim et al., 2012; Menrad and Menssen, 2005). The aptide specific for EDB (APT_{EDB}) was conjugated with liposome to form an APT_{EDB}-liposome complex, and siRNA was encapsulated within this APT_{EDB}-liposome complex (FIG. 15G). We confirmed the successful delivery of APT_{EDB}-liposome into LLC tumor tissues (arrowhead, FIG. 15H) and the knockdown of RhoJ with encapsulated RhoJ siRNA (en-siJ) compared to encapsulated control siRNA (en-siC) (FIGS. 15I-K). In vivo experiments showed that the anti-tumor effect of en-siJ (2 mg/kg) monotherapy or VEGF-trap (25 mg/kg) monotherapy was similar, showing a 40-50% decrease in tumor volume compared to the control group, while the combination therapy of en-siJ with VEGF-trap increased this effect to 66% (FIG. 7D). Moreover, en-siJ or VEGF-trap monotherapy decreased tumor vessel densities by ˜45% and ˜50% in the peri- and intratumoral regions, respectively, but the combination therapy showed a 66% and 68% respective reduction (FIGS. 7E and 7F). Notably, intratumoral hemorrhage of en-siJ-treated tumors dramatically decreased with VEGF-trap treatment (FIGS. 7G and 7H), indicating that RhoJ blockade induces vascular disruption and hemorrhage in a VEGF-dependent manner, which is consistent with findings of the in vitro permeability assay (FIG. 6J). Finally, the combination therapy showed a 75% reduction in LN metastasis, which was greater than either en-siJ or VEGF-trap monotherapy (FIGS. 71 & 7J). Together, the dual blockade of RhoJ and VEGF signaling is superior to the single blockade in anti-tumor, anti-angiogenic, and anti-metastatic activity.

### Example 2.10

### RhoJ Blockade Augments the Anti-Tumor Effect of a VDA, Combretastatin-A4-Phosphate (CA4P)

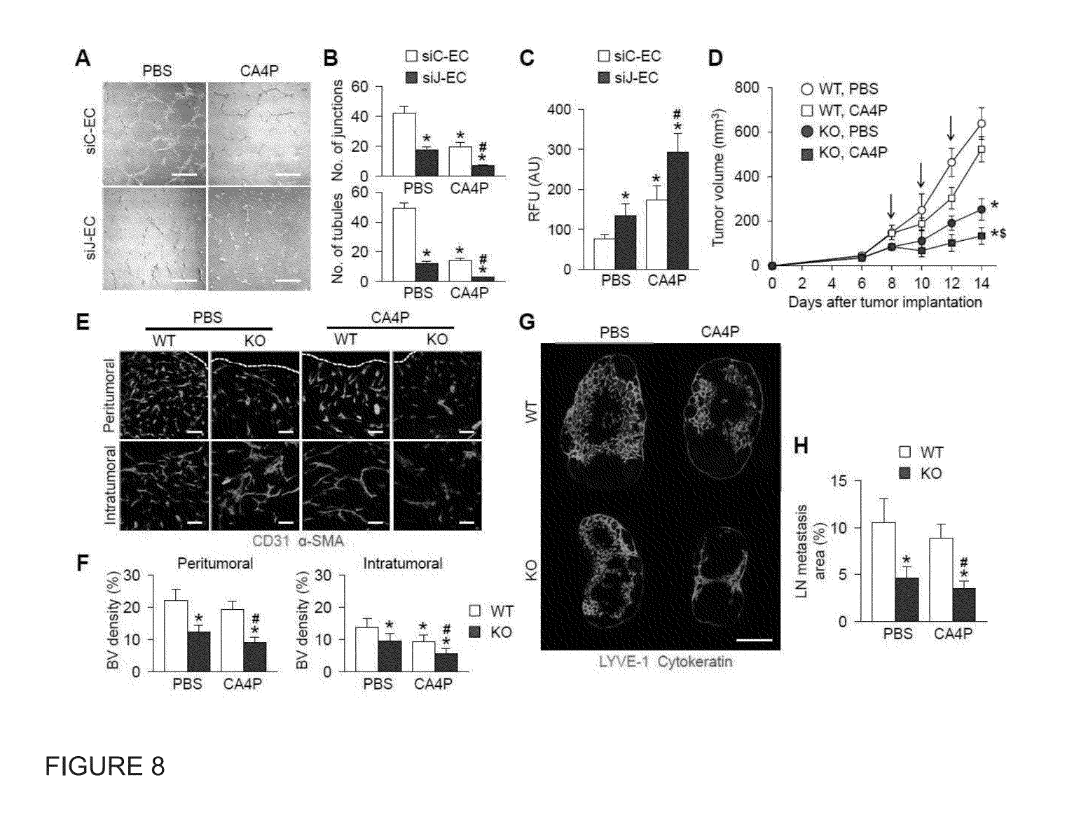

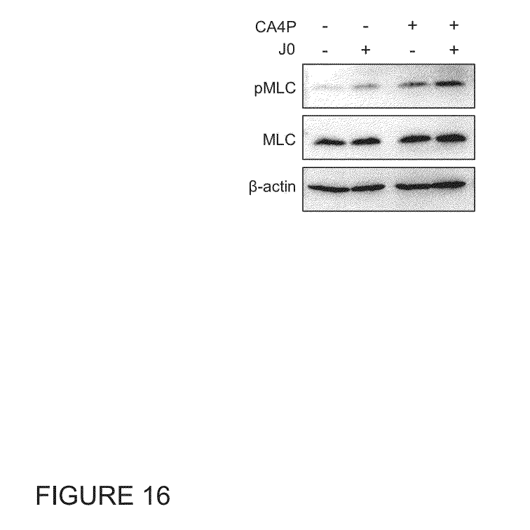

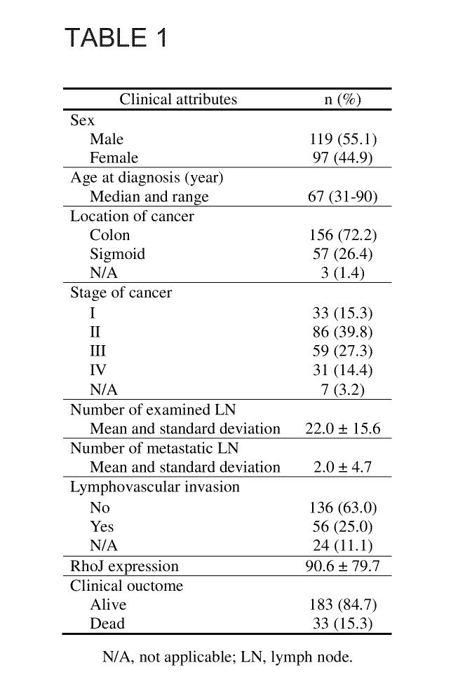

VDAs are known to disrupt established tumor vessels by directly targeting the cytoskeletons of ECs (Siemann, 2011). Because RhoJ blockade is comparable to VDAs in inducing tumor vascular disruption, we speculated that RhoJ blockade might have an enhancing effect with VDAs, such as CA4P. The in vitro tube formation assay revealed that RhoJ knockdown in concert with CA4P (20 nM) treatment profoundly inhibited EC tube formation, inducing almost complete disruption, compared to single treatment with either CA4P or RhoJ siRNA (FIGS. 8A and 8B). Also, permeability across the EC monolayer of siJ-ECs treated with CA4P was increased by 2.9-fold compared to that of siC-ECs treated with PBS (FIG. 8C). Moreover, RhoJ knockdown in combination with CA4P further activated RhoA-ROCK signaling pathway (FIG. 16). These suggest a possible collaboration between RhoJ blockade and VDA treatments through complementation and enhancement of the vascular-disrupting effect. To confirm this hypothesis, we evaluated the influence of CA4P on RhoJ-WT and KO mice. Treatment with CA4P (50 mg/kg) resulted in only an 18% reduction in tumor growth of WT mice, in which the response to CA4P was not maintained and tumors began to regrow. However, RhoJ-KO mice displayed a 79% additional inhibition in tumor growth when treated with CA4P, in which a durable response to CA4P was observed. (FIG. 8D). Moreover, CA4P reduced vascular densities by 59% and 60% in the peri- and intratumoral regions of RhoJ-KO mice, respectively, which was more potent compared to the respective 13% and 31% reduction in RhoJ-WT mice (FIGS. 8E and 8F). Finally, CA4P displayed an efficient anti-metastatic effect in RhoJ-KO mice, reducing metastasis by 67%, but no reduction in RhoJ-WT mice (FIGS. 8G and 8H). Taking these data together, we could confirm that RhoJ blockade is a valuable complementary therapy to overcome the limitations of current VDA therapy.

### REFERENCES

Baguley, B. C., and McKeage, M. J. (2012). Anticancer potential of tumor vascular disrupting agents: review of the latest clinical evidence. Clinical Investigation 2, 985-993.
Beckers, C. M. L., van Hinsbergh, V. W. M., and van Nieuw Amerongen, G. P. (2010). Driving Rho GTPase activity in endothelial cells regulates barrier integrity. Thrombosis & Haemostasis 19, 40.
Bergers, G., and Hanahan, D. (2008). Modes of resistance to anti-angiogenic therapy. Nature Reviews Cancer 8, 592-603.
Bono, F., De Smet, F., Herbert, C., De Bock, K., Georgiadou, M., Fons, P., Tjwa, M., Alcouffe, C., Ny, A., Bianciotto, M., et al. (2013). Inhibition of Tumor Angiogenesis and Growth by a Small-Molecule Multi-FGF Receptor Blocker with Allosteric Properties. Cancer cell 23, 477-488.
Bryan, B., and d'Amore, P. (2007). What tangled webs they weave: Rho-GTPase control of angiogenesis. Cellular and Molecular Life Sciences 64, 2053-2065.
Carmeliet, P., and Jain, R. K. (2011). Molecular mechanisms and clinical applications of angiogenesis. Nature 473, 298-307.
Casanovas, O, Hicklin, D. J., Bergers, G., and Hanahan, D. (2005). Drug resistance by evasion of antiangiogenic targeting of VEGF signaling in late-stage pancreatic islet tumors. Cancer cell 8, 299-309.
Chen, H. X., and Cleck, J. N. (2009). Adverse effects of anticancer agents that target the VEGF pathway. Nature Reviews Clinical Oncology 6, 465-477.
Chung, A. S., Lee, J., and Ferrara, N. (2010). Targeting the tumour vasculature: insights from physiological angiogenesis. Nature Reviews Cancer 10, 505-514.
Ebos, J. M., and Kerbel, R. S. (2011). Antiangiogenic therapy: impact on invasion, disease progression, and metastasis. Nature Reviews Clinical Oncology 8, 210-221.
Ellis, L. M., and Hicklin, D. J. (2008). VEGF-targeted therapy: mechanisms of anti-tumour activity. Nature Reviews Cancer 8, 579-591.
Ferrara, N., and Alitalo, K. (1999). Clinical applications of angiogenic growth factors and their inhibitors. Nature medicine 5, 1359.
Ferrara, N., and Kerbel, R. S. (2005). Angiogenesis as a therapeutic target. Nature 438, 967-974.
Fukumura, D., and Jain, R. K. (2007). Tumor microvasculature and microenvironment: targets for anti-angiogenesis and normalization. Microvascular research 74, 72-84.
Fukushima, Y., Okada, M., Kataoka, H., Hirashima, M., Yoshida, Y., Mann, F., Gomi, F., Nishida, K., Nishikawa, S. I., and Uemura, A. (2011). Sema3E-PlexinD1 signaling selectively suppresses disoriented angiogenesis in ischemic retinopathy in mice. The Journal of Clinical Investigation 121, 1974.
Gerald, D., Chintharlapalli, S., Augustin, H. G., and Benjamin, L. E. (2013). Angiopoietin-2: An Attractive Target for Improved Antiangiogenic Tumor Therapy. Cancer research 73, 1649-1657.
Hanahan, D., and Folkman, J. (1996). Patterns and emerging mechanisms of the angiogenic switch during tumorigenesis. Cell 86, 353.
Hanahan, D., and Weinberg, R. A. (2011). Hallmarks of cancer: the next generation. Cell 144, 646-674.
Horsman, M. R., and Siemann, D. W. (2006). Pathophysiologic effects of vascular-targeting agents and the implications for combination with conventional therapies. Cancer research 66, 11520-11539.
Joo, H. J., Choi, D.-K., Lim, J. S., Park, J.-S., Lee, S.-H., Song, S., Shin, J. H., Lim, D.-S., Kim, I., and Hwang, K.-C. (2012). ROCK suppression promotes differentiation and expansion of endothelial cells from embryonic stem cell-derived Flk1+ mesodermal precursor cells. Blood 120, 2733-2744.
Kamba, T., and McDonald, D. (2007). Mechanisms of adverse effects of anti-VEGF therapy for cancer. British journal of cancer 96, 1788-1795.
Kaur, S., Leszczynska, K., Abraham, S., Scarcia, M., Hiltbrunner, S., Marshall, C. J., Mavria, G., Bicknell, R., and Heath, V. L. (2011). RhoJ/TCL regulates endothelial motility and tube formation and modulates actomyosin contractility and focal adhesion numbers. Arteriosclerosis, Thrombosis, and Vascular Biology 31, 657-664.
Kim, S., Kim, D., Jung, H. H., Lee, I. H., Kim, J. I. L., Suh, J. Y., and Jon, S. (2012). Bio-Inspired Design and Potential Biomedical Applications of a Novel Class of High-Affinity Peptides. Angewandte Chemie International Edition 51, 1890-1894.
Koh, Y. J., Kim, H.-Z., Hwang, S.-I., Lee, J. E., Oh, N., Jung, K., Kim, M., Kim, K. E.,
Kim, H., and Lim, N.-K. (2010). Double antiangiogenic protein, DAAP, targeting VEGF-A and angiopoietins in tumor angiogenesis, metastasis, and vascular leakage. Cancer cell 18, 171-184.
Lakso, M., Pichel, J. G., Gorman, J. R., Sauer, B., Okamoto, Y., Lee, E., Alt, F. W., and Westphal, H. (1996). Efficient in vivo manipulation of mouse genomic sequences at the zygote stage. Proceedings of the National Academy of Sciences of the United States of America 93, 5860-5865.
Leszczynska, K., Kaur, S., Wilson, E., Bicknell, R., and Heath, V. L. (2011). The role of RhoJ in endothelial cell biology and angiogenesis. Biochemical Society Transactions 39, 1606.
Li, B., and Dewey, C. (2011). RSEM: accurate transcript quantification from RNA-Seq data with or without a reference genome. BMC bioinformatics 12, 323.
Mancuso, M. R., Davis, R., Norberg, S. M., O'Brien, S., Sennino, B., Nakahara, T., Yao,
V. J., Inai, T., Brooks, P., and Freimark, B. (2006). Rapid vascular regrowth in tumors after reversal of VEGF inhibition. Journal of Clinical Investigation 116, 2610-2621.
Masiero, M., SimOes, Filipa C., Han, Hee D., Snell, C., Peterkin, T., Bridges, E., Mangala, Lingegowda S., Wu, Sherry Y.-Y., Pradeep, S., Li, D., et al. (2013). A Core Human Primary Tumor Angiogenesis Signature Identifies the Endothelial Orphan Receptor ELTD1 as a Key Regulator of Angiogenesis. Cancer cell 24, 229-241.
McDonald, D. M., and Baluk, P. (2002). Significance of blood vessel leakiness in cancer. Cancer research 62, 5381-5385.
Menrad, A., and Menssen, H. D. (2005). ED-B fibronectin as a target for antibody-based cancer treatments. Expert Opinion on Therapeutic Targets 9, 491-500.
Rodriguez, C. I., Buchholz, F., Galloway, J., Sequerra, R., Kasper, J., Ayala, R., Stewart, A. F., and Dymecki, S. M. (2000). High-efficiency deleter mice show that FLPe is an alternative to Cre-loxP. Nature genetics 25, 139-140.
Saw, P. E., Ko, Y. T., and Jon, S. (2010). Efficient Liposomal Nanocarrier-mediated Oligodeoxynucleotide Delivery Involving Dual Use of a Cell-Penetrating Peptide as a Packaging and Intracellular Delivery Agent. Macromolecular rapid communications 31, 1155-1162.
Sawada, J., Urakami, T., Li, F., Urakami, A., Zhu, W., Fukuda, M., Li, D. Y., Ruoslahti, E., and Komatsu, M. (2012). Small GTPase R-Ras Regulates Integrity and Functionality of Tumor Blood Vessels. Cancer cell 22, 235-249.
Schiller, M. R. (2006). Coupling receptor tyrosine kinases to Rho GTPases—GEFs what's the link. Cellular signalling 18, 1834-1843.
Sennino, B., and McDonald, D. M. (2012). Controlling escape from angiogenesis inhibitors. Nature Reviews Cancer 12, 699-709.
Shojaei, F., Wu, X., Malik, A. K., Zhong, C., Baldwin, M. E., Schanz, S., Fuh, G., Gerber, H.-P., and Ferrara, N. (2007). Tumor refractoriness to anti-VEGF treatment is mediated by CD11b+ Gr1+ myeloid cells. Nature biotechnology 25, 911-920.
Siemann, D. W. (2011). The unique characteristics of tumor vasculature and preclinical evidence for its selective disruption by tumor-vascular disrupting agents. Cancer treatment reviews 37, 63-74.
Sun, H., Breslin, J. W., Zhu, J., Yuan, S. Y., and Wu, M. H. (2006). Rho and ROCK Signaling in VEGF-Induced Microvascular Endothelial Hyperpermeability. Microcirculation 13, 237-247.
Takase, H., Matsumoto, K., Yamadera, R., Kubota, Y., Otsu, A., Suzuki, R., Ishitobi, H., Mochizuki, H., Kojima, T., and Takano, S. (2012). Genome-wide identification of endothelial cell-enriched genes in the mouse embryo. Blood 120, 914-923.
Tozer, G. M., Kanthou, C., and Baguley, B. C. (2005). Disrupting tumour blood vessels. Nature Reviews Cancer 5, 423-435.
Trédan, O., Galmarini, C. M., Patel, K., and Tannock, I. F. (2007). Drug resistance and the solid tumor microenvironment. Journal of the National Cancer Institute 99, 1441-1454.
Tvorogov, D., Anisimov, A., Zheng, W., Leppanen, V.-M., Tammela, T., Laurinavicius, S., Holnthoner, W., Heloterä, H., Holopainen, T., and Jeltsch, M. (2010). Effective suppression of vascular network formation by combination of antibodies blocking VEGFR ligand binding and receptor dimerization. Cancer cell 18, 630-640.
Uesaka, T., Jain, S., Yonemura, S., Uchiyama, Y., Milbrandt, J., and Enomoto, H. (2007). Conditional ablation of GFRalphal in postmigratory enteric neurons triggers unconventional neuronal death in the colon and causes a Hirschsprung's disease phenotype. Development 134, 2171-2181.
van der Meel, R., Symons, M. H., Kudernatsch, R., Kok, R. J., Schiffelers, R. M., Storm, G., Gallagher, W. M., and Byrne, A. T. (2011). The VEGF/Rho GTPase signalling pathway: a promising target for anti-angiogenic/anti-invasion therapy. Drug discovery today 16, 219-228.
Wachsberger, P., Burd, R., and Dicker, A. P. (2003). Tumor Response to Ionizing Radiation Combined with Antiangiogenesis or Vascular Targeting Agents Exploring Mechanisms of Interaction. Clinical Cancer Research 9, 1957-1971.
Wang, Y., Nakayama, M., Pitulescu, M. E., Schmidt, T. S., Bochenek, M. L., Sakakibara, A., Adams, S., Davy, A., Deutsch, U., and Lüthi, U. (2010). Ephrin-B2 controls VEGF-induced angiogenesis and lymphangiogenesis. Nature 465, 483-486.
Wang, K., Singh, D., Zeng, Z., Coleman, S. J., Huang, Y., Savich, G. L., He, X., Mieczkowski, P., Grimm, S. A., and Perou, C. M. (2010). MapSplice: accurate mapping of RNA-seq reads for splice junction discovery. Nucleic acids research 38, e178-e178.
Yuan, L., Sacharidou, A., Stratman, A. N., Le Bras, A., Zwiers, P. J., Spokes, K., Bhasin, M., Shih, S., Nagy, J. A., and Molema, G. (2011). RhoJ is an endothelial cell-restricted Rho GTPase that mediates vascular morphogenesis and is regulated by the transcription factor ERG. Blood 118, 1145-1153.
Yagi, T., Tokunaga, T., Furuta, Y., Nada, S., Yoshida, M., Tsukada, T., Saga, Y., Takeda, N., Ikawa, Y., and Aizawa, S. (1993). A novel ES cell line, TT2, with high germline-differentiating potency. Analytical biochemistry 214, 70-76.
Zhou, Z., Wang, J., Cao, R., Morita, H., Soininen, R., Chan, K. M., Liu, B., Cao, Y., and Tryggvason, K. (2004). Impaired angiogenesis, delayed wound healing and retarded tumor growth in perlecan heparan sulfate-deficient mice. Cancer research 64, 4699-4702.

All of the references cited herein are incorporated by reference in their entirety.

Those skilled in the art will recognize, or be able to ascertain using no more than routine experimentation, many equivalents to the specific embodiments of the invention specifically described herein.

What is claimed is:

1. A method of inhibiting tumor growth comprising contacting the tumor with a compound that inhibits activity of RhoJ protein.

2. The method according to claim 1, wherein the tumor growth occurs in a subject.

3. The method according to claim 2, wherein the subject is a mammal.

4. The method according to claim 3, wherein the subject is human.

5. A method of inhibiting cancer metastasis in a subject comprising administering to the subject a compound that inhibits activity of RhoJ protein.

6. The method according to claim 5, wherein the subject is a mammal.

7. The method according to claim 6, wherein the subject is human.

8. A method of reducing tumor volume comprising contacting the tumor with a compound that inhibits activity of RhoJ protein.

9. The method according to claim 8, wherein the tumor volume reduction occurs in a subject.

10. The method according to claim 9, wherein the subject is a mammal.

11. The method according to claim 10, wherein the subject is human.

12. The method according to claim 1, wherein the compound is an oligonucleotide complementary to a portion of RhoJ transcript, an antagonistic ligand of RhoJ, or a chemical compound that inhibits the activity of RhoJ.

13. The method according to claim 1, comprising further contacting the tumor with or administering to the subject, a compound that sequesters VEGF in combination with the compound that inhibits activity of RhoJ protein.

14. The method according to claim 13, wherein the compound that sequesters VEGF is VEGF-trap.

15. The method according to claim 1, comprising further contacting the tumor with or administering to the subject, a vascular-disrupting agent (VDA) in combination with the compound that inhibits activity of RhoJ protein.

16. The method according to claim 15, wherein the VDA is combretastatin-A4-phosphate (CA4P).

17. The method according to claim 1, comprising further contacting the tumor with or administering to the subject, a cytotoxic therapeutic agent in combination with the compound that inhibits activity of RhoJ protein.

18. The method according to claim 17, wherein the cytotoxic therapeutic agent is cisplatin.

19. The method according to claim 1, wherein the compound or agent is included in a carrier.

20. The method according to claim 19, wherein the carrier is an aptide conjugated liposome.
